(19) United States
(12) Patent Application Publication
(10) Pub. No.: US 2024/0189344 A1
(43) Pub. Date: Jun. 13, 2024

(54) METHODS USING CARBON DIOXIDE AND OXYGEN TO IMPROVE CHARACTERISTICS OF POULTRY

(71) Applicants: Sameer H. Israni, Darien, IL (US); Balazs Hunek, Western Springs, IL (US)

(72) Inventors: Sameer H. Israni, Darien, IL (US); Balazs Hunek, Western Springs, IL (US)

(21) Appl. No.: 18/064,406

(22) Filed: Dec. 12, 2022

(51) Int. Cl. A61K 33/00; A01K 41/04; A61P 21/00

(52) U.S. Cl. CPC A61K 33/00; A01K 41/04; A61P 21/00

(57) ABSTRACT

Disclosed are methods of reducing incidence of disorders such as Woody Breast in poultry, by incubating pre-hatching eggs in an atmosphere enriched in carbon dioxide, preferably combined with administration to the post-hatching birds of water comprising oxygen-containing nanobubbles.

### FIELD OF THE INVENTION

The present invention relates to treatment of poultry, especially chickens, to improve the characteristics of the chicken from a time at or after hatching of the egg and thereafter as the chick grows to a grown adult. The characteristics that may be improved reduced or eliminated incidence of a condition known as Woody Breast in the hatched, grown chicken.

### BACKGROUND OF THE INVENTION

A large proportion of modern chickens that are grown to be sources of their meat to be consumed, especially the ones grown in the US, are grown to relatively large sizes (heavier than 6 pounds). The large size is believed to be desirable as it provides breast meat in relatively large quantities per chicken. Chickens grown for their meat are called “broilers”. Chickens grown to larger sizes (heavier than 6 pounds) for their meat are sometimes referred to as “big birds”.

However, chickens of these large sizes, especially heavier than 6 pounds, are often found to have a condition called “Woody Breast” or “Wooden Breast” (referred to herein as Woody Breast or WB). In this condition the breast muscles (which provide the breast meat) have a very hard texture and lower nutritional value. These characteristics substantially reduce the value of the breast meat. It has been estimated that on average 5-20% of big birds (6 pounds or more in weight at the time of slaughter) suffer from Woody Breast, resulting in undesirable overall losses to the chicken growers. Woody Breast is exhibited in the live chicken, before any processing or cooking of the chicken for its meat, and is not prevented or reduced by any processing or cooking of the breast meat. The search has been ongoing for a solution for treating or avoiding Woody Breast condition, other than reducing the size to which the chickens are grown, which itself would reduce their overall value.

### BRIEF SUMMARY OF THE INVENTION

One aspect of the present invention comprises a method of reducing or eliminating the incidence of woody breast in poultry that has hatched from a fertilized unhatched poultry egg, comprising, before the poultry has hatched from the egg, incubating the egg for an incubation period of 18 to 21 consecutive days in a gaseous atmosphere which is in contact with the egg, and during that time, feeding carbon dioxide from a source outside the egg into the gaseous atmosphere as necessary so that for at least one period of time of at least 12 hours the carbon dioxide concentration in the gaseous atmosphere which is in contact with the egg is 7,500 ppm to 20,000 ppm.

This aspect of the present invention identifies specific treatment regimens applying carbon dioxide (CO₂) concentrations and regimens (or “recipes”) to unhatched fertilized embryonic poultry eggs while they are being incubated, that in the case of chicken eggs have been found to be successful in reducing Woody Breast in chickens hatched from the treated eggs, and in promoting the other characteristics mentioned herein (namely increased hatchability of the egg; increased hatch weight of the chicken; increased viability and decreased mortality of the chicken; and increased weight gain of the growing chicken). These effects have been found with the regimens described herein of established CO₂ concentrations in the incubator for established periods of time. These regimens are all practically and safely implementable in commercial hatcheries, without causing any other harmful side effects such as reduced hatchability, increased mortality, or other disease conditions, to the hatched chickens.

The present invention can be characterized as a method of incubating a fertilized unhatched chicken or other poultry egg to hatching, comprising incubating the egg for an incubation period of 18 to 21 consecutive days in a gaseous atmosphere which is in contact with the egg, and during that time, injecting carbon dioxide from a source outside the egg into the gaseous atmosphere as necessary so that for at least one period of time (or, alternatively, at least six periods of time) of at least 12 hours (or, alternatively, of at least 24 hours) the carbon dioxide concentration in the gaseous atmosphere which is in contact with the egg is 7,500 ppm to 20,000 ppm.

The inventors have identified several incubating regimens, including the following:
Regimen 1: The fertilized unhatched chicken or other poultry egg is exposed for at least one day, preferably at least 6 days, and more preferably at least 12 days of the first 18 days of the incubation period to a gaseous atmosphere which is in contact with the egg and which contains carbon dioxide at an incubation concentration that is within 10%, and preferably within 5%, of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10%, and preferably within 5%, of a set value between 7,500 ppm CO₂ and 20,000 ppm CO₂.

In a preferred embodiment of this Regimen 1, the set value throughout the first 18 days is constant for all 18 days.
Regimen 2: In a second preferred regimen, the fertilized unhatched chicken or other poultry egg is exposed to a gaseous atmosphere which is in contact with the egg for consecutive incremental periods in each of which incremental periods the carbon dioxide concentration in the gaseous atmosphere is maintained at a value that is within 10%, and preferably within 5%, of a set value that is constant throughout the incremental period and is between the CO₂ concentration of the ambient atmosphere (which is considered to be on the order of 350 ppm to as high as 420 ppm CO₂) and 20,000 ppm CO₂ by feeding carbon dioxide into the gaseous atmosphere from a source outside the egg as necessary to maintain the CO₂ concentration in the gaseous atmosphere within 10%, and preferably within 5%, of said set value, wherein the set values and the CO₂ concentrations in the gaseous atmosphere increase from each of said incremental periods to the next incremental period, such that in the last incremental period the CO₂ concentration in the gaseous atmosphere is within 10%, and preferably within 5%, of a value between 7,500 ppm CO₂ and 20,000 ppm CO₂, and then for 3 consecutive days immediately following the first 18 days of the incubation period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10%, and preferably within 5%, of a constant set value between 7,500 ppm CO₂ and 20,000 ppm CO₂.

In a preferred embodiment of Regimen 2, each set value increases from one incremental period to the next incremental period linearly, that is, by equal increments.
Regimen 3: A third preferred regimen comprises, throughout the incubation period, alternatingly
   (A) exposing the fertilized unhatched chicken or other poultry egg to a gaseous atmosphere which is in contact with the egg for an exposure period, in each of which exposure periods the carbon dioxide concentration in the gaseous atmosphere is maintained at a value that is within 10%, and preferably within 5%, of a set value between 7,500 ppm CO₂ and 20,000 ppm CO₂ that is constant in each exposure period, by feeding carbon dioxide into the gaseous atmosphere from a source outside the egg as necessary to maintain the CO₂ concentration in the gaseous atmosphere in the exposure period within 10%, and preferably within 5%, of said set value, wherein the set value is the same in all of the exposure periods, and
   (B) exposing the fertilized unhatched egg for an intervening period to a gaseous atmosphere into which no carbon dioxide is fed into the gaseous atmosphere from outside the egg.

In preferred aspects of this regimen, each exposure period is 18 to 24 hours in duration and each intervening period is 18 to 24 hours in duration; and there are 2 to 20 exposure periods and 2 to 20 intervening periods.
Regimen 4: A fourth preferred regimen comprises, throughout the incubation period, alternatingly
   (A) exposing the fertilized unhatched chicken or other poultry egg to a gaseous atmosphere which is in contact with the egg for an exposure period, in each of which exposure periods the carbon dioxide concentration in the gaseous atmosphere is maintained at a value that is within 10%, and preferably within 5%, of a set value between the CO₂ concentration of the ambient atmosphere and 20,000 ppm CO₂ that increases from one exposure period to the next exposure period, by feeding carbon dioxide into the gaseous atmosphere from a source outside the egg as necessary to maintain the CO₂ concentration in the gaseous atmosphere in the exposure period within 10%, and preferably within 5%, of said set value, such that in the last incremental period the CO₂ concentration in the gaseous atmosphere is within 10%, and preferably within 5%, of a value between 7,500 ppm CO₂ and 20,000 ppm CO₂ and
   (B) exposing the fertilized unhatched egg for an intervening period to a gaseous atmosphere into which no carbon dioxide is fed into the gaseous atmosphere from outside the egg.

In preferred aspects of this fourth regimen, each exposure period is 18 to 24 hours in duration and each intervening period is 18 to 24 hours in duration.

Another aspect of the present invention comprises methods of reducing or eliminating the incidence of woody breast in a chicken or other poultry that has hatched from a fertilized unhatched poultry egg, comprising, before the poultry has hatched from the egg, incubating the egg for an incubation period of 18 to 21 consecutive days in a gaseous atmosphere which is in contact with the egg, and during that time, feeding carbon dioxide from a source outside the egg into the gaseous atmosphere as necessary so that for at least one period of time of at least 12 hours the carbon dioxide concentration in the gaseous atmosphere which is in contact with the egg is 7,500 ppm to 20,000 ppm,

and then
administering enterally to the chicken water comprising in the water nanobubbles which comprise oxygen. In preferred embodiments of the present invention, the nanobubbles comprise at least 21 vol. % oxygen, preferably 50 vol. % to 100 vol. % oxygen, more preferably 90 vol. % to 100 vol. % oxygen, and yet more preferably at least 99.0 vol. % oxygen.

In other preferred embodiments of this aspect of the present invention, the average diameter of said nanobubbles is 10 to 1000 nanometers, preferably 10 to 400 nanometers, more preferably 30 to 200 nanometers, and yet more preferably 70 to 130 nanometers.

In yet other preferred embodiments of this aspect of the present invention, the water administered to the chicken comprises at least 100 million of said nanobubbles per milliliter of water, preferably at least 200 million of said nanobubbles per milliliter of water, and more preferably at least 500 million of said nanobubbles per milliliter of water.

In another preferred embodiment of this aspect of the present invention, the aggregate amount of oxygen in said nanobubbles and dissolved in the water administered to the chicken is to 150 milligrams of oxygen per liter of said water, preferably 35 to 100 milligrams of oxygen per liter of said water, and more preferably 60 to 100 milligrams of oxygen per liter of said water.

In this aspect of the present invention, 50 vol. % to 100 vol. % and preferably 90 vol. % to 100 vol. % of the water and more preferably at least 95 vol. % or even 99 vol. % to 100 vol. % of the water that is made available to the chicken to drink contains oxygen-containing nanobubbles as described herein. The provision for administration of water containing oxygen-containing nanobubbles as described herein preferably begins upon hatching of the chicken from its egg, but can begin up to 2 days and even up to 2 weeks from hatching.

In this aspect of the invention the pressure on the drinking water containing the oxygen-containing nanobubbles should be above atmospheric pressure, preferably at least 1 psig and up to 25 psig until the bird ingests the water.

Advantageously, in this aspect of the present invention the nanobubbles that are administered in water to the chicken are absorbed through the gastrointestinal system of the chicken into the circulatory system of the chicken.

As used throughout this description and the claims herein, “ppm” means parts per million on a weight basis.

As used throughout this description and the claims herein, the term “ambient atmosphere” means the atmosphere in the region inside and immediately outside the incubator.

As used throughout this description and the claims herein, the term “as necessary” means that carbon dioxide is added if the CO₂ concentration of the atmosphere into which the CO₂ is to be added is below a desired range or level, and that no CO₂ is added if the CO₂ concentration in the atmosphere is at a level that is within the desired range or is at a desired level.

As used throughout this description and the claims herein, “carbon dioxide from a source outside the egg” excludes carbon dioxide that has entered the incubator from the natural atmosphere outside the incubator and excludes carbon dioxide that has passed out of the egg through the shell of the egg from inside the egg.

As used throughout this description and the claims herein, “relative humidity” of air or gas refers to the amount of water vapor currently present in the air or gas as percentage of maximum amount of water vapor that the air or gas can hold (without causing precipitation) at the given air or gas temperature. When maximum water vapor capacity of the air or gas, at the given temperature, is reached or exceeded, i.e. when relative humidity reaches 100%, water vapor will precipitate out as liquid water. Many different devices are easily available and used by those skilled in the art to measure relative humidity in the air or gas.

As used throughout this description and the claims herein, the term “incubation” refers to the period when fertilized eggs are placed in a controlled atmosphere environment which is warm enough to promote embryo growth and development. Typically, in commercial hatcheries, the first 18 days of incubation take place in a room or equipment that is referred to as either the setter or the incubator and the process is usually referred to as setting. Then the developed eggs are transferred to a separate room or equipment called the hatcher and the process is usually referred to as hatching. As used throughout this description and the claims herein, the term “incubation” refers to the combination of the setting and hatching phases.

In most commercial chicken hatcheries, the setting period is usually 18 days and the hatching period is usually 3 days. In some cases, these periods can be different. The present invention also applies if the setting and hatching periods are different than what is described throughout this description and the claims herein. For example, if the total period is 25 days rather than 21 days, the present invention would still apply. In this case, either by feeding carbon dioxide into the gaseous atmosphere from a source outside the egg for 18 days or even extending the period proportionally to 22 days.

The present invention can also apply for incubation of poultry species other than chicken. As used herein, “poultry” means chicken, turkey, duck, and goose. Also, “poultry” is used herein to refer to a single bird, or to a plurality of birds, depending on the context in which the word “poultry” is used. For example, the present invention would also apply to turkey egg incubation. Typically, turkey egg incubation periods are longer than chicken egg incubation periods. As in the previous paragraph the CO₂ regimens described in this invention can be kept the same or the time periods can be extended proportionally.

The present invention provides methods, recipes and equipment for the incubation under controlled CO₂ concentrations of unhatched fertilized embryonic eggs of chickens to increase the respiratory and vascular system development and respiratory and vascular function of the chickens before and after hatching, with all other incubation conditions and controls being comparable. In commercial incubating methods and equipment that do not practice the present invention, the CO₂ concentration evolves in an uncontrolled and varying fashion naturally or by happenstance due to limiting otherwise conditioned air circulation in the incubator. The present invention discovered the criticality of controlling the CO₂ concentration and specific regimens of concentration and timing in the incubator atmosphere to hatch and grow healthy chickens exhibiting reduced, or no, incidence of Woody Breast, and exhibiting the other characteristics mentioned herein (increased hatchability; increased hatch weight; increased viability and decreased mortality; and increased weight gain).

### BRIEF DESCRIPTION OF THE DRAWINGS

FIG. 1 is a plan view of an incubator and associated apparatus with which the present invention can be practiced.

FIGS. 2A, 2B, 2C, and 2D are graphs illustrating several different regimens for establishing CO₂ atmospheres in the incubator.

FIG. 3A is a bar graph of Woody Breast Scores for an example of regimen 1. FIGS. 3B, 3C, 3D, 3E are corresponding results for hatchability, hatch weight, final weight and livability of the birds subjected to regimen 1 compared to control birds.

FIG. 4A is a bar graph of Woody Breast Scores for an example of regimen 2. FIGS. 4B, 4C, 4D, 4E are corresponding results for hatchability, hatch weight, final weight and livability of the birds subjected to regimen 2 compared to control birds.

FIG. 5A is a bar graph of Woody Breast Scores for an example of regimen 3. FIGS. 5B, 5C, 5D, 5E are corresponding results for hatchability, hatch weight, final weight and livability of the birds subjected to regimen 3 compared to control birds.

FIG. 6 is a flowsheet showing apparatus useful in performing the present invention.

FIGS. 7 and 8 are bar graphs of nanobubble concentrations in water.

FIG. 9 is a bar graph of scores of the extent of woody breast for groups of chickens.

FIG. 10 is a bar graph of scores of the extent of woody breast for other groups of chickens.

### DETAILED DESCRIPTION OF THE INVENTION

The aspects of the present invention that involve incubation in atmospheres that contain carbon dioxide are advantageously carried out with any fertilized poultry eggs, preferably fertilized chicken eggs, and especially with fertilized eggs that are expected, upon hatching, to produce chickens that are capable with appropriate care and feeding to grow into chickens exhibiting weight of at least 6 pounds, preferably at least 6.5 pounds, and even at least 8 or 10 pounds, by 42 to 58 days after hatching. Such eggs are preferably obtained from chickens that are genetically favored to produce eggs which will hatch to produce chickens of such sizes.

The aspects of this invention that involve incubation under specific conditions in atmospheres that contain carbon dioxide, can be carried out in an incubator that is largely of conventional design and features, and that is provided with the capability of feeding carbon dioxide into the incubator, and with the capability of measuring the concentration of carbon dioxide in the atmosphere within the incubator and of initiating and discontinuing the feeding of carbon dioxide into the incubator in response to measured values of the concentration of carbon dioxide within the incubator.

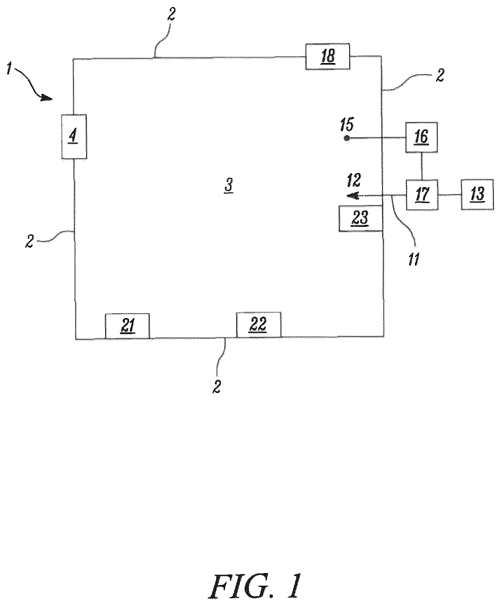

For example, referring to FIG. 1, an incubator useful in the practice of this invention comprises a structure 1 that houses an enclosed space 3 that is defined by walls 2 and by a floor (not shown) and a ceiling (not shown) which are sealed to the walls, and that contains at least one entry 4 through which persons and trolleys or racks with eggs can pass into and out of space 3 to place eggs in the incubator and to remove eggs or hatched chicks out of the incubator. The vertical distance from floor to ceiling is typically on the order of six feet or more in larger structures typically used by commercial scale producers, which enable persons to stand inside the incubator, but smaller structures can be used too. Typically, the walls are insulated, and usually the floor and ceiling are also insulated, to help hold heat within space 3 in aid of the incubation of eggs within the incubator.

Gas line 11 ending at outlet 12 inside space 3 conveys gaseous carbon dioxide or a gas composition that contains carbon dioxide through controllable valve 17 from gas source 13 which can be a storage tank, cylinder, vessel or container, or a delivery truck, which contains carbon dioxide at a delivery concentration that is typically at least 80 vol. % CO₂. One or more probe(s) 15 measures the carbon dioxide concentration inside space 3. Monitor 16 controls the opening and closing of valve 17 in line 11 in response to the values measured by probe 15, by comparing the measured concentration values to preprogrammed values in monitor 16 of the concentration of CO₂ that is to be maintained in space 3. When the measured value of the carbon dioxide concentration is below a predetermined desired value stored in the monitor 16, the monitor opens valve 17 so that carbon dioxide is fed into space 3 until the measured value of the CO₂ concentration in the gaseous atmosphere in space 3 has increased to the desired value. Carbon dioxide can be fed by any of numerous ways, including (but not limited to) feeding a gaseous stream of 100% carbon dioxide, or feeding a gaseous stream that is a combination (mixture) of carbon dioxide together with one or more other gaseous substances, or feeding liquid or solid carbon dioxide, or adding a substance that can generate or release carbon dioxide into the gaseous atmosphere. When the measured carbon dioxide concentration in space 3 reaches the desired value, as detected by probe 15 and monitor 16, monitor 16 closes valve 17. Exhaust port 18 represents any suitable opening, openable and closable on demand, through which atmosphere can be vented out of space 3. The incubator should preferably also include equipment that provides the ability to measure the amounts of fresh or conditioned air (from outside the incubator) flowing into the incubator, and/or the flow rate at which the atmosphere within the incubator leaves the incubator. This measurement can then be used to determine the flow rate of CO₂ feeding required to maintain a certain concentration of CO₂.

To lower the CO₂ concentration in the atmosphere to which the egg is exposed in the incubator, one can feed air, or oxygen and/or nitrogen, into the atmosphere while removing atmosphere from the interior of the incubator, whereby the amount of CO₂ in the atmosphere relative to other gaseous components present is lowered.

The incubator is also equipped with air or gas temperature control (which can be either or both of a heater, and/or cooler such as an air conditioner unit 21 and with a source 22 of water vapor such as a steam line or an evaporator, all of which are known and conventionally available, and each of which are equipped with suitable controls to activate and deactivate the supply of heat, cooling and of humidity, respectively, so as to controllably maintain the atmosphere in space 3 at desired values of temperature and relative humidity. The incubator typically comprises a number of racks on which the eggs to be incubated are placed.

Most commercial incubators contain an internal fan 23 that distributes or mixes the air inside the incubator, ensuring uniform temperature and humidity conditions. Location of carbon dioxide gas outlet 12 within the incubator is important relative to the location of the fan to ensure uniform distribution of the fed CO₂ gas. Outlet 12 of the carbon dioxide gas line can be composed of either a single point or injection nozzle or alternatively it can be composed of multiple nozzles or points of injection. It is important to locate the outlet 12 close to the fan 23. A preferred configuration is a ring of nozzles that inject CO₂ gas very close to the internal fan inside the incubator, either right upstream or downstream of the fan 21. Preferably the outlet 12 ring of nozzles has a diameter matching the diameter of the fan blades and located 2 to 12 inches, either upstream or downstream, from the fan.

The position of probe 15 is also important to ensure uniform and accurate distribution of the fed CO₂ gas. Preferably the probe 15 is located at least the distance of half the incubator width away from outlet 12. Also the probe 15 should be located at the center of one of the egg racks, most preferably only 1 to 3 inches away from the surface of the eggs at the center of the rack. All of this ensures that the probe 15 is measuring the right CO₂ environment within the incubator and is not producing false readings. A multitude of CO₂ probes can be used for large incubation chambers, or for chambers with inadequate gas mixing. Average reading or some other means of utilizing spatially distributed CO₂ readings can be used to control CO₂ addition in these cases.

In operation, the gaseous atmosphere in the incubator should also include at least 17 vol. % oxygen, more preferably more than 19% and most preferably more than 20 vol. % oxygen concentration

These components, and the amounts of each component, can be provided in the incubator by known techniques of adding and measuring the desired components of the overall gaseous atmosphere.

The temperature of the gaseous atmosphere in which the eggs are incubated should be in the range of 92° F. to 103° F. The relative humidity level in the gaseous atmosphere should be 30% to 70% relative humidity.

Eggs being incubated need a warm (typically 100° F.) temperature and controlled humidity for optimum growth. However, the eggs themselves generate heat and expel moisture and generate carbon dioxide during the incubation process. The amounts of heat, moisture and carbon dioxide put out by the fertilized eggs (or developing embryos) vary throughout the incubation process and can also be different from one genetic strain to another. Thus, the ability to control the amount of fresh air that is allowed to enter the incubator (accompanied by the same amount of air from within the incubator that exits it) varies depending on the strain of the birds, number of eggs inside the incubator and time during the incubation process.

Some previous studies restricted the fresh air exchanges into the incubator (non-ventilation) to increase the amount of carbon dioxide inside the incubator to approximately 7,000 ppm. This is an available method to increase the carbon dioxide levels inside the incubator. This method can be practiced by completely turning off the fresh air exchanges or modulating the fresh air exchanges based on incubator carbon dioxide concentration. However, this non-ventilation method, does not allow the operator to simultaneously control the carbon dioxide levels while also independently controlling the temperature and humidity, which can lead to negative effects on the hatchability of the eggs, mortality of the resulting chicken, disease incidences, low weight gain, higher food consumption etc. In most commercial incubators, more importance is given to controlling temperature and humidity levels inside the incubator. This results in a variable and uncontrolled carbon dioxide level during the incubation process.

The present invention provides a methodology for controlling the carbon dioxide levels inside the incubator without restricting or changing the amount of fresh air exchanges that is done in conventional incubators. This is achieved by feeding carbon dioxide into the incubator from an external carbon dioxide source in a controlled fashion based on measurements of carbon dioxide levels inside the incubator. Thus, as a result, carbon dioxide levels can be controlled independent of the temperature and humidity. This results in the most optimum temperature, humidity and carbon dioxide environment for egg incubation.

The incubation of chicken eggs in typical commercial practice does not employ the controlled CO₂ atmospheres according to the present invention, and thus does not add any CO₂ from an external source. As a result, the concentration of CO₂ in the incubator atmosphere varies widely during the multi-day course of the incubation; CO₂ concentration levels can and do change during the incubation between values as widely separated as 500 to 7,000 ppm. The CO₂ concentration changes throughout the 18 to 21 days of incubation and hatching, and there is no pattern to the change. Some CO₂ is generated by the egg/embryo itself as part of its respiratory process and enters the incubator atmosphere through the eggshell. Overall, in such commercial incubator operations, the concentration of CO₂ in the incubator atmosphere is influenced only by the exchanges of conditioned air to control temperature, oxygen and humidity levels, and not by the deliberate feeding of CO₂ to maintain a desired CO₂ level during incubation. The embryos subjected to these conditions of varying CO₂ concentration levels typically exhibit significant levels of Woody Breast.

To carry out the incubation method according to the present invention, one or more fertilized unhatched eggs are placed into the incubator, typically onto a rack in space 3 (seen in FIG. 1). The desired temperature and humidity levels are established in space 3 by conditioning the air entering the incubator, by carrying out air exchange as needed between the atmospheres inside and outside the incubator, and by heating, cooling, humidifying, and dehumidifying the atmosphere in the incubator using conventional equipment situated within or in fluid communication with the incubator for performing each of said functions. Then, carbon dioxide concentration according to any of the regimens described herein, is established in the gaseous atmosphere in space 3 to which the egg(s) being incubated are exposed.

### Regimen 1:

Beginning at a point in time from fertilization up to four weeks following fertilization, the fertilized unhatched egg is placed in the incubator and is kept in the incubator in contact with a gaseous atmosphere in the incubator that contains carbon dioxide at an incubation concentration that is within 10%, and preferably within 5%, of a set value between 7,500 ppm CO₂ and 20,000 ppm CO₂ for a period of at least 12 days of the next 18 consecutive days, preferably all 18 days. Preferably, the incubation concentration of carbon dioxide is within 10%, and preferably within 5%, of a set value between 7,500 ppm CO₂ and 15,000 ppm CO₂. More preferably, the incubation concentration is maintained at within 10%, and preferably within 5%, of 10,000 ppm CO₂. The set value can be varied during the 18-day period, but the set value is preferably constant throughout the 18-day period. During the 18-day period, carbon dioxide is fed into the atmosphere in the incubator as necessary to maintain the CO₂ concentration in which the egg is incubated at the desired incubation concentration.

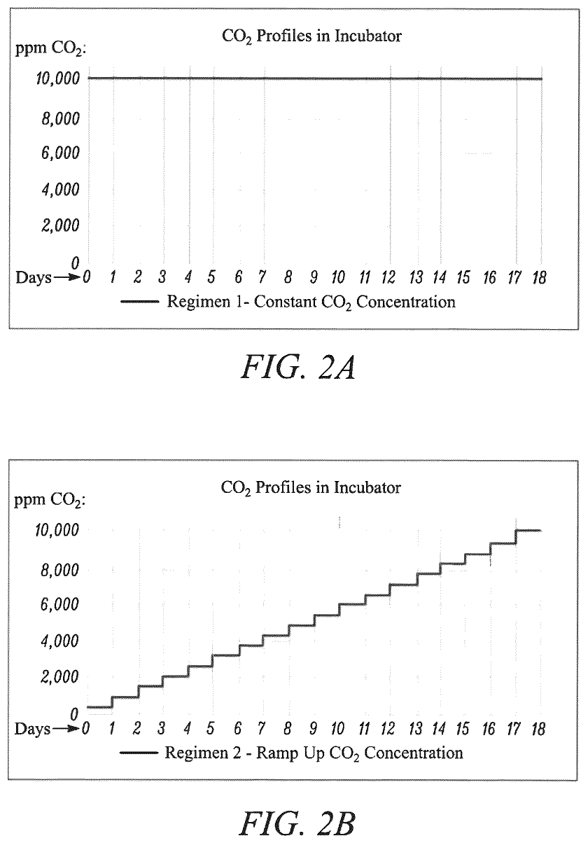

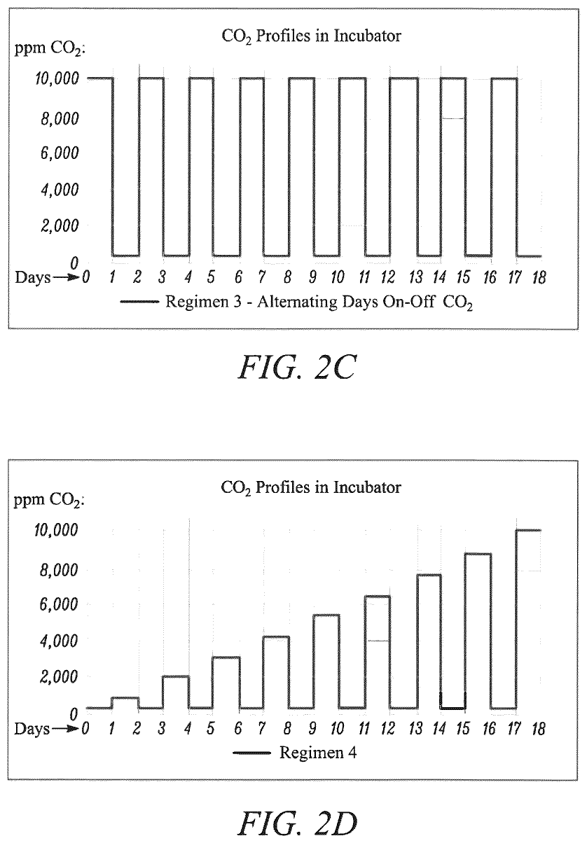

FIG. 2A illustrates one embodiment of this regimen, in which the concentration of CO₂ is maintained at a constant value of 10,000 ppm for the full extent of 18 days of incubation of the egg.

At the end of the 18-day period, the egg can be removed to a hatching room, or it can be kept in the incubator, for a total of up 3 additional days. During that period of up to 3 additional days, one may choose between (a) not feeding any additional CO₂ from any source outside the egg into the atmosphere to which the egg is exposed (thereby permitting the atmosphere to contain CO₂ naturally present in air and any CO₂ that is generated by the embryos and passes through the shell into the incubator atmosphere); and (b) feeding CO₂ as necessary from any source outside the egg so that the atmosphere to which the egg is exposed contains carbon dioxide at a concentration of up to within 10%, and preferably within 5%, of a set value that is between 7,500 ppm CO₂ and 20,000 ppm CO₂ (preferably, between 7,500 ppm CO₂ and 15,000 ppm CO₂, more preferably 10,000 ppm).

### Regimen 2:

Beginning at a point in time from fertilization up to four weeks following fertilization, the fertilized unhatched egg is placed in the incubator in contact with a gaseous atmosphere in the incubator that contains carbon dioxide at an incubation concentration which follows the following pattern:

For up to 18 consecutive periods (termed “incremental periods” herein) of 18 to 24 hours each, the carbon dioxide concentration in the gaseous atmosphere is maintained at a value that is within 10%, and preferably within 5%, of a set value between 350 ppm CO₂ and 20,000 ppm CO₂. In each successive incremental period, the set value increases, so that the actual carbon dioxide concentration in the incubator is higher than what it was in the immediately preceding incremental period. Preferably, the set value increases from one incremental period to the next by equal increments.

Carbon dioxide is fed into space 3 as necessary to increase the carbon dioxide concentration in the incubator to the next desired higher value. The carbon dioxide concentration is established and maintained at the desired value by feeding carbon dioxide into the gaseous atmosphere from a source outside the egg (such as through outlet 12) as necessary to maintain the CO₂ concentration in the gaseous atmosphere within 10%, and preferably within 5%, of said set value. Preferably, in the last incremental period, the carbon dioxide concentration in the atmosphere is within 10%, and preferably within 5%, of a set value between 7,500 ppm and 20,000 ppm CO₂ and more preferably within 10%, and preferably within 5%, of a set value between 7,500 ppm and 15,000 ppm or even more preferably within 10% and preferably within 5% of 10,000 ppm CO₂. Thus, the set values in the 24-hour periods near the beginning of this regimen should not be near 20,000 ppm CO₂ or even 15,000 ppm CO₂ or even 10,000 ppm or even 7,500 ppm CO₂.

FIG. 2B illustrates an embodiment of this regimen. The distance between each pair of consecutive numbers on the x-axis (such as 0 to 1, 1 to 2, 2 to 3, and so on) represents the passage of one 24-hour period. In the embodiment illustrated in FIG. 2B, the CO₂ concentration is maintained at a constant level during each 24-hour period, and from one 24-hour period to the very next 24-hour period the CO₂ concentration in the incubator is increased to a higher level which is then held constant throughout that next 24-hour period. In this embodiment, the CO₂ concentration in the incubator is about 350 ppm during the first 24-hour period, and is 10,000 ppm during the eighteenth 24-hour period. FIG. 2B illustrates an embodiment in which the CO₂ concentration in the incubator is increased by the same increment from each 24-hour period to the next, though increasing the CO₂ concentration in the incubator by equal increments is not necessary in the practice of this invention.

At the end of the up to 18 periods of up to 24 hours, the egg can be removed to a hatching room, or it can be kept in the incubator, for a total of up 3 additional days. During that period of up to 3 additional days, one may choose between (a) not feeding any additional CO₂ from any source outside the egg into the atmosphere to which the egg is exposed (thereby permitting the atmosphere to contain CO₂ naturally present in air and any CO₂ that is generated by the embryos and passes through the shell into the incubator atmosphere); and (b) feeding CO₂ as necessary from any source outside the egg so that the atmosphere to which the egg is exposed contains carbon dioxide at a concentration of up to within 10%, and preferably within 5%, of a set value that is between 7,500 ppm CO₂ and 20,000 ppm CO₂ (preferably, between 7,500 ppm CO₂ and 15,000 ppm CO₂, more preferably up to 10,000 ppm).

### Regimen 3:

Beginning at a point in time from fertilization up to four weeks following fertilization, the fertilized unhatched egg is placed in the incubator in contact with a gaseous atmosphere in the incubator that contains carbon dioxide at an incubation concentration which follows the following pattern:

The carbon dioxide concentration in the gaseous atmosphere is alternated between values for what is termed the exposure periods, and values for what is termed the intervening periods. In the exposure periods, the carbon dioxide concentration is maintained at a value that is within 10%, and preferably within 5%, of a set value between 7,500 ppm CO₂ and 20,000 ppm (preferably 15,000 ppm or even up to 10,000 ppm) of CO₂ by feeding carbon dioxide into the gaseous atmosphere from a source outside the egg as necessary to maintain the CO₂ concentration in the gaseous atmosphere within 10%, and preferably within 5%, of said set value. The set value is the same in all of the first 24-hour periods of each 48-hour period.

Alternatingly between exposure periods, in the intervening periods, no carbon dioxide is fed into the gaseous atmosphere of the incubator from outside the egg. The carbon dioxide concentration in the gaseous atmosphere in space 3 may decrease during this intervening period, or it may increase (or have its decrease offset) by carbon dioxide that passes into the space 3 from within the egg, through the intact but naturally microporous shell. However, for the intervening periods, the CO₂ concentration in the incubator can be reduced to a desired low level, as low as ambient, by opening vents that permit the atmosphere within the incubator to pass out of the incubator (optionally activating fans or blowers to facilitate movement of the atmosphere out of the incubator) and feeding ambient air at sufficient levels to establish only the lower desired CO₂ concentration level inside the incubator.

FIG. 2C illustrates an embodiment of this regimen. In this embodiment, consecutive 48-hour periods are represented by the space from 0 to 2 and then by the spaces between consecutive even numbers (that is, 2 to 4, 4 to 6, and so on, through 16-18). Within each 48-hour period that is depicted in this way, the first 24-hour period is the interval extending from the lower even number, to the next odd number to the right, and the second 24-hour period is the interval extending from that odd number to the next even number to the right. For example, in the 48-hour period that is depicted as extending from 4 to 6, the first 24-hour period extends from 4 to 5 and the second 24-hour period extends from 5 to 6.

In the embodiment illustrated in FIG. 2C, sufficient CO₂ will have been fed into the incubator atmosphere so that the CO₂ concentration in the incubator is 10,000 ppm in the 24-hour period from 0 to 1 (this being an exposure period), and CO₂ will have been vented out of the incubator so that the CO₂ concentration in the incubator atmosphere is as low as ambient in the 24-hour period from 1 to 2 (this being an intervening period). Then, sufficient CO₂ is injected into the incubator atmosphere so that the CO₂ concentration in the incubator is 10,000 ppm in the 24-hour exposure period from 2 to 3, following which addition of CO₂ into the incubator is discontinued and the CO₂ concentration in the incubator is again lowered to as low as ambient in the 24-hour intervening period from 3 to 4. This pattern is repeated alternatingly for the duration of the regimen. The establishment of CO₂ concentrations in the incubator alternating between 10,000 ppm and as low as ambient in consecutive 24-hour periods is continued in the same manner through the eighteenth 24-hour period. Depending on the number of eggs incubated at the same time in the incubator, the CO₂ levels on the alternate days when no external or additional CO₂ is fed, may increase above the normal ambient value, since the eggs themselves generate CO₂.

At the end of the ninth, tenth or eleventh 48-hour period, the egg can be removed to a hatching room, or it can be kept in the incubator, for a total of up 21 days from when the egg was first placed into the incubator. During that period following the end of the ninth 48-hour period, one may choose to add no carbon dioxide into the incubator from any source outside the egg, or one may choose to feed carbon dioxide from any source outside the egg to maintain a concentration in the incubator atmosphere of up to 20,000 ppm CO₂ and preferably up to 15,000 ppm or even 10,000 ppm CO₂.

### Regimen 4:

Beginning at a point in time from fertilization up to four weeks following fertilization, the fertilized unhatched egg is placed in the incubator in contact with a gaseous atmosphere in the incubator that contains carbon dioxide at an incubation concentration which follows the following pattern:

The carbon dioxide concentration in the gaseous atmosphere is alternated between values for what is termed the exposure periods, and values for what is termed the intervening periods. In the exposure periods, the carbon dioxide concentration is maintained at a value that is within 10%, and preferably within 5%, of a set value between the CO₂ concentration in the ambient atmosphere and 20,000 ppm of CO₂ by feeding carbon dioxide into the gaseous atmosphere from a source outside the egg as necessary to maintain the CO₂ concentration in the gaseous atmosphere within 10%, and preferably within 5%, of said set value. The CO₂ concentration and the set value in each exposure period is higher than the set value and CO₂ concentration in the preceding exposure periods, such that in the last incremental period the CO₂ concentration in the gaseous atmosphere is within 10%, and preferably within 5%, of a value between 7,500 ppm CO₂ and 20,000 ppm CO₂.

Alternatingly between exposure periods, in the intervening periods, no carbon dioxide is fed into the gaseous atmosphere of the incubator from outside the egg. The carbon dioxide concentration in the gaseous atmosphere in space 3 may decrease during this intervening period, or it may increase (or have its decrease offset) by carbon dioxide that passes into the space 3 from within the egg, through the intact but naturally microporous shell. However, for the intervening periods, the CO₂ concentration in the incubator can be reduced to a desired low level such as the CO₂ concentration in the ambient atmosphere by opening vents that permit the atmosphere within the incubator to pass out of the incubator (optionally activating fans or blowers to facilitate movement of the atmosphere out of the incubator) and feeding air at sufficient levels to establish only the lower desired CO₂ concentration level inside the incubator.

FIG. 2D illustrates an embodiment of this regimen 4. In this embodiment, consecutive 48-hour periods are represented by the space from 0 to 2 and then by the spaces between consecutive even numbers (that is, 2 to 4, 4 to 6, and so on, through 16-18). Within each 48-hour period that is depicted in this way, the intervening period is the interval extending from zero or from the lower even number, to the next odd number to the right, and the exposure period is the interval extending from that odd number to the next even number to the right. For example, in the 48-hour period that is depicted as extending from 4 to 6, the intervening period extends from 4 to 5 and the exposure period extends from 5 to 6.

In the embodiment illustrated in FIG. 2D, CO₂ will have been vented out of the incubator so that the CO₂ concentration in the incubator atmosphere is around 400 ppm in the 24-hour period from 0 to 1 (this being an intervening period). Then sufficient CO₂ will have been fed into the incubator atmosphere so that the CO₂ concentration in the incubator is 10,000 ppm in the 24-hour period from 1 to 2 (this being an exposure period). After CO₂ is vented from the incubator to establish the intervening period from 2 to 3, sufficient CO₂ is fed into the incubator atmosphere so that the CO₂ concentration in the incubator is 10,000 ppm in the 24-hour exposure period from 3 to 4, following which addition of CO₂ into the incubator is discontinued and the CO₂ concentration in the incubator is again lowered to around 400 ppm in the 24-hour intervening period from 4 to 5. This pattern is repeated alternatingly for the duration of the regimen. The establishment of CO₂ concentrations in the incubator alternating between 10,000 ppm and around 400 ppm in consecutive 24-hour periods is continued in the same manner through the eighteenth 24-hour period. Depending on the number of eggs incubated at the same time in the incubator, the CO₂ levels on the alternate days when no external or additional CO₂ is added, may increase above the normal atmospheric value of around 400 ppm, since the eggs themselves generate CO₂.

At the end of the ninth, tenth or eleventh 48-hour period, the egg can be removed to a hatching room, or it can be kept in the incubator, for a total of up 21 days from when the egg was first placed into the incubator. During that period following the end of the ninth 48-hour period, one may choose to add no carbon dioxide into the incubator from any source outside the egg, or one may choose to feed carbon dioxide from any source outside the egg to maintain a concentration in the incubator atmosphere between 7,500 ppm to 20,000 ppm CO₂ and preferably between 7,500 ppm to 15,000 ppm or more preferably at 10,000 ppm CO₂.

Establishing a lower CO₂ concentration in any of the respective intervening periods can be achieved in the same manner as described above with respect to Regimen 3.

In each of the foregoing regimens 1 through 4, where carbon dioxide concentrations are expressed as within 10%, and preferably within 5%, of a given value, it is more preferred that the concentrations are within 1% (instead of within 10% or 5%) of the given value.

At the end of any regimen of exposing the egg to carbon dioxide-containing atmospheres as described above, a chick is thereafter hatched from the thus treated and incubated egg. The chick is then fed water and food and is grown into a fully-grown chicken, under conventional practices by which chicks are fed and enabled to grow into fully-grown chickens.

As shown in the Example below, the aforementioned regimens of exposure of the egg to an atmosphere containing prescribed concentrations of carbon dioxide, for prescribed periods of time, have been found to promote growth of chickens to full size, even to sizes in excess of 6 pounds or in excess of 6.5 pounds, with reduced or no incidence of Woody Breast in the chicken.

The aforementioned benefits in addition to reduction of incidence of Woody Breast, namely increased hatchability of the egg; increased hatch weight of the chicken; increased viability and decreased mortality of the chicken; increased weight gain of the growing chicken, have also been found with the regimens described herein of established CO₂ concentrations in the incubator for established periods of time.

Procedures that incorporate any of these regimens have additional advantages. They are practically implementable in commercial hatcheries while still allowing hatchery personnel to enter the incubators for short periods, as they normally do to perform regular tasks, without endangering the personnel on account of excessive or prolonged exposure to an atmosphere containing unsafe levels of carbon dioxide. In addition, these regimens do not cause other harmful side-effects to the chicken through its life cycle. Thus, these regimens do not cause reduction in rate of growth, final weight, % hatchability, % livability, or susceptibility to other disorders or diseases.

When the poultry chicks have hatched, they should be removed from the carbon dioxide-enriched atmosphere (either by opening the incubation chamber to the ambient atmosphere, or, preferably, by physically moving the chicks to another location). Then, the incidence of woody breast in the chicks as they grow can be further reduced or prevented by feeding them drinking water that contains nanobubbles that contain oxygen, as described herein.

In this aspect of the invention, nanobubbles are bubbles less than 1 micron is diameter and preferably less than 400 microns in diameter, and more preferably less than 250 microns in size. The nanobubbles of a size within a range as described herein, and containing oxygen at a concentration within a range as described herein, and maintained at a pressure within a range as described herein, fed to the chickens as described herein, have been found to be successful in significantly reducing WB incidences.

Forming in water the nanobubbles having the desired characteristics described herein, involves the following:

Oxygen is added into the drinking water that will be fed to the chickens to form oxygen-enriched nanobubbles (by which is meant nanobubbles having oxygen in a content higher than the oxygen content of atmospheric air.) The drinking water is typically well water or city water, but can be from any source of potable water.

Any of several known methods can be employed to generate in the water the desired oxygen-enriched nanobubbles having the desired oxygen content, size, and concentration. There exist multiple methods of generating oxygen-enriched nanobubbles, e.g. (1) dissolving oxygen gas into water at high pressure, then de-pressurizing the water at rate that is controlled to ensure that nanobubbles of oxygen are generated during the de-pressurization; (2) using a venturi based gas liquid mixing device that controls the size and number of bubbles generated; or (3) passing oxygen-containing gas through a nanoporous membrane (with or without a surface coating) to create very small bubbles in the liquid on the discharge side of the membrane. For the purposes of this invention, the technique (1) of pressure-dissolution described above was used and is preferred.

The concentration of oxygen in the gas used to make the nanobubbles should be higher than in air, preferably at least 90 vol. % (with the balance remaining being primarily other inert gases like nitrogen and argon), and more preferably should be more than 99.9 vol. %, and most preferably should be more than 99.99% purity oxygen.

The mean diameter of stable nanobubbles generated should be between 30 to 200 nanometers (nm), as measured by NanoSight analysis (NTA, available commercially through Malvern Panalytical). Preferably the mean diameter should be between 70 to 130 nm.

The amount of oxygen nanobubbles generated should be at least 100 million nanobubbles per milliliter (ml) of the water in which the nanobubbles are formed, preferably above 200 million bubbles per ml, and most preferably above 500 million bubbles per ml of the water. A useful way to measure the number generated of oxygen-enriched nanobubbles is described below. If NTA is used as the method of analysis then the first step is to measure the concentration of nanobubbles in the original drinking water before it is oxygenated. This measured concentration is referred to as Nᵢₙᵢₜᵢₐₗ. This Nᵢₙᵢₜᵢₐₗ includes nanobubbles that contain air air, and may also contain other solid nanoparticles that do not provide any benefits for the purposes of this invention. Then the drinking water is oxygenated with oxygen-enriched nanobubbles as described herein, and a sample of this water is analyzed by NTA. The measured concentration of nanobubbles in this sample is N_{final}. The actual number of generated oxygen-enriched nanobubbles is then calculated as N_{O2NB}=N_{final}−Nᵢₙᵢₜᵢₐₗ. For the practice of the present invention, this N_{O2NB} should be at least 100 million oxygen-enriched nanobubbles per ml of water containing the nanobubbles, preferably above 200 million oxygen-enriched nanobubbles per ml, and most preferably above 500 million oxygen-enriched nanobubbles per ml of drinking water.

The dissolved oxygen (DO) measured in the drinking water should be between 20 to 45 ppm of oxygen. This is measured using a DO probe by taking a sample of the drinking water with the generated oxygen-enriched nanobubbles and measuring the DO at atmospheric pressure. DO probes only measure the dissolved oxygen portion of the oxygen added to the drinking water. DO probes do not measure the amount of oxygen added to generate the oxygen nanobubbles.

The water that is used to administer the oxygen-enriched nanobubbles should be oxygenated to a level between 20 to 150 mg of total added oxygen per liter of water and more preferably between 35 to 100 mg/l, most preferably between 60 to 100 mg/l. The total added oxygen is measured as the total oxygen present in the water which includes the dissolved oxygen plus the oxygen present in the oxygen-enriched nanobubbles. The amount of oxygen present in the oxygen-enriched nanobubbles can be measured by using a sodium sulfite titration methodology. Throughout the process of generation of oxygen-enriched nanobubbles and even after the generation of the desired quantity of oxygen-enriched nanobubbles, the water containing the nanobubbles should be maintained under higher than atmospheric pressure.

Techniques for forming nanobubbles in water, wherein the characteristics are as desired herein such as the number and size of the nanobubbles in the water, are known in the technical literature such as U.S. Pat. No. 10,293,309 and the U.S. patent application published under publication number US2007/0286795A1. Devices for forming nanobubbles in water, that are useful in performing the present invention, are known and available commercially.

Several prior art documents specify adding chemical components that enhance the stability of the nanobubbles in the water. For example, the U.S. patent application published under publication number US2007/0286795A1 specifies increasing the salinity of the water up to 3.5%. However, for the purpose of drinking water, these additional chemicals can cause adverse effects on birds. It is highly desirable to avoid adding any additional chemicals to the drinking water besides the oxygen rich gas. An important step in the current invention is to maintain slight pressure on the nanobubble containing drinking water. In the absence of chemicals to enhance the stability of the nanobubbles, this slight positive pressure maintains the stability of the nanobubbles, providing the Woody Breast reduction efficacy. While not preferred, the water that contains the nanobubbles can contain other substances, such as medications, nutrients etc, provided that any substances present in the water do not adversely affect the health of the chicken.

The water that contains the nanobubbles should be at a temperature of no more than 5 to degrees F. above room temperature, the better to protect against the oxygen being released from the water. Preferably the temperature of the water is 35 to 125 degrees F., more preferably 50 to 100 degrees F. Accordingly, provision can be included in the apparatus described with respect to FIG. 1 to cool or heat the water as needed in order to prevent the temperature of the water from increasing undesirably.

The drinking water containing oxygen-enriched nanobubbles as described herein is administered by any of several possible methodologies. For instance, a human operator or handler can insert into each chicken's mouth the end of a dropper or syringe filled with the water, and feed the water out of the end of the dropper or syringe into the chicken's mouth or throat.

A preferable mode of administering to the chicken the drinking water containing oxygen-enriched nanobubbles is to provide the water in a way that the chicken can drink the water entirely by its own actions, whenever it wants to drink water, without needing intervention of another person (other than, possibly, demonstration to the chicken of how to gain access to the water, when the chicken is first put in proximity to the supply of the water containing the oxygen-enriched nanobubbles). Preferably, in this arrangement, all of the water that is available for the chicken to drink contains oxygen-enriched nanobubbles as described herein. However, benefits of the present invention are available (though perhaps to a smaller extent) if 5 vol. % or more of the water that is available for the chicken to drink contains the oxygen-containing nanobubbles as described herein; a greater benefit is realized if at least 50 vol % and more preferably 90 vol. % of the water that is available for the chicken to drink contains the oxygen-containing nanobubbles, and even more preferably at least 99 vol. % of the water that is available for the chicken to drink contains the oxygen-containing nanobubbles.

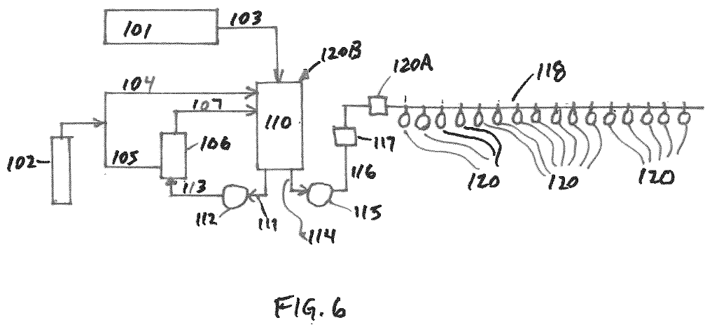

A preferred apparatus with which the present invention may be performed is shown in FIG. 6, in which can be seen source 101 of water to be oxygenated with the nanobubbles, and source 102 of oxygen which is preferably a cylinder or similar dispenser that contains oxygen that is preferably at a purity of at least 95 vol. % and preferably at least 99 vol. %. Water flows through line 103 from water source 101 to tank 110. Oxygen flows through line 104 from oxygen source 102 to tank 110, and through line 105 from oxygen source 102 to nanobubble generator 106 which can be any commercially available device able to generate nanobubbles in the water to the extent and with the characteristics desired as described herein. Water containing nanobubbles as described herein flows through line 107 from nanobubble generator 106 into tank 110. Water containing nanobubbles as described herein can be recycled from tank 110 through line 111 and recirculating pump 112 into line 113 which feeds the water into nanobubble generator 106. Water containing the nanobubbles as described herein is fed out of tank 110 in line 114 through pump 115 into line 116 which conveys the water containing the oxygen-containing nanobubbles to one or more devices 118 such as nipples 120 which hold the water containing the oxygen-enriched nanobubbles and which do not permit water to pass out of the device 118 or the nipples 120 except when the chicken taps the device (i.e. a nipple 120) with its beak. Optional cooler 117 can be placed in line 116 to maintain the temperature of the water in line 116 at a level that is not so high that evolution of oxygen from the water occurs. Alternatively, the cooler 117 could be placed inside tank 110, in line 113 or other similar places. Preferably, pressurized nipple drinkers on the vessel containing the water should be used to ensure that the water remains pressurized until a few seconds before the chicken drinks it. The pressure on the drinking water containing the oxygen-containing nanobubbles at this stage should be above atmospheric pressure, preferably at least 1 psig and up to 25 psig until the bird ingests the water. Typically this is achieved by using commercially available nipple type drinkers, that include a component 120A which maintains the water in a pressurized state. Pressure on the water that contains the oxygen-containing nanobubbles can also be maintained by a suitable component 120B that applies pressure to the water in tank 110. Pressure on the water line can also be maintained by the pump 115.

The birds tap on one of several nipples 120 when they want water. The tapping temporarily opens the nipple 120 allowing one or more drops of water containing oxygen-enriched nanobubbles to come out and be consumed by the chicken. An alternative apparatus includes a bowl that is open to the atmosphere and that contains water containing oxygen-enriched nanobubbles.

The oxygen-enriched nanobubbles in the drinking water should be replenished at regular intervals to ensure that the concentration does not drop below the specified value of 100 million bubbles per ml of water. Preferably, the replenishment is carried out at least every 12 hours. This means that the equipment for oxygenating the water with oxygen-enriched nanobubbles should be run at least every 12 hours to make sure that the level of oxygen-enriched nanobubbles present in the drinking water remains above 100 million bubbles per ml, preferably above 200 million bubbles per ml, and most preferably above 500 million bubbles per ml of drinking water.

The chicken will benefit from drinking the oxygen-enriched drinking water for even small periods of time at any stage of their growth. Preferably, the drinking water with oxygen-enriched nanobubbles should be fed to the chickens either immediately upon hatching or at the most starting from no longer than 2 weeks, more preferably no longer than 2 days, after hatching. Typically, the drinking water is available to the chickens on demand, i.e. whenever they want without restriction. From the start point, the drinking water containing oxygen-enriched nanobubbles can continue to be fed to the chicken everyday for different periods of time, preferably at a minimum for one week from the start of the administration, but most preferably for the entire growing period until the chickens are fully grown. Typically commercial broilers are grown for a period of 6 to 9 weeks from hatch. Thus the total period during which the chicken is fed water containing oxygen-enriched nanobubbles should be between 1 week up to the entire lifetime after hatching.

The main benefit of the above methodology is a 50 to 100% reduction in WB scores for broiler chicken that drink the water containing oxygen-enriched nanobubbles. An example of the benefit is described in the Examples below. Other potential benefits of the technology include better health, lower mortality, higher weight gains, more efficient feed conversion, and better meat quality. This methodology can also be employed with any other living species in addition to broiler chickens.

The WB benefits apply the most to broilers that are going to be grown to a slaughter weight of 6 pounds or higher, and more preferably 6.5 pounds or higher. Only certain genetic lines of birds are chosen to be grown to be larger than 6.5 pounds in weight. Other benefits apply to all category of broiler and potentially all living species.

### Example 1

This example describes regimens of incubation in the presence of increased and controlled concentrations of CO₂ that have been found to show reduction in incidence or severity of Woody Breast in chickens.

### Example—Regimen 1

### Experimental:

900 fertilized eggs from a commonly used commercial chicken strain, Ross 708, that were less than 1 week old since being laid, and laid in the same laying facility by layers (chickens) of the same age, were split equally into three batches of 300 each. One batch, referred to as control batch, was incubated for 18 days in an atmosphere in which no additional CO₂ was injected into this atmosphere from any source outside the egg. For this batch, the CO₂ concentration inside the incubator varied between the CO₂ concentration of the ambient atmosphere (i.e. about 409 ppm) and as high as 1,500 ppm (resulting from CO₂ generation by the eggs themselves). The second batch, referred to as 4,000 ppm test CO₂ batch or 0.4% CO₂ test batch, was incubated for 18 days wherein the CO₂ concentration in the incubator from the start of the first day through the end of the eighteenth day was maintained at 4,000 ppm (i.e. 0.4% CO₂) by occasional injections of CO₂ as necessary. The third batch, referred to as 10,000 ppm test CO₂ batch or 1% CO₂ test batch, was incubated for 18 days according to Regimen 1 described herein, wherein the CO₂ concentration in the incubator from the start of the first day through the end of the eighteenth day was maintained at 10,000 ppm (i.e. 1% CO₂) by occasional injections of CO₂ as necessary. All three batches were incubated at approximately 100° F. and between 30% and 70% relative humidity. At the end of the eighteenth day of incubation, all three batches of eggs were transferred to hatcher equipment, where the eggs hatched over the next 3 days. No external CO₂ was injected into the hatcher from any source outside the eggs for this period of 3 days. Thus, the only difference between the conditions under which the three batches were incubated was that in the control batch no additional CO₂ was injected, whereas for the other two batches additional, external CO₂ was injected to subject the eggs to CO₂ concentrations of 4,000 ppm and 10,000 ppm respectively. During the incubation period the oxygen concentration in the control incubator was always above 20%, whereas in the 4,000 ppm and 10,000 ppm CO₂ controlled incubators, the oxygen level was typically above 19%.

Once the chicks hatched, approximately 150 of the male chicks from each batch were taken to grow cages and they began to be fed water and feed. From this point onwards, there was no difference in the treatment of the birds. 28 days after hatching the chickens from each batch were weighed and then stunned. Then the birds were measured for Woody Breast scores by manual palpitation by a trained panel of experts under single-blinded conditions. Each breast was given a score of either 0, 1, 2 or 3. Score 0 means no WB was detected. Score 3 meant very high levels of WB detected and scores 1 and 2 meant intermediate levels of WB were detected.

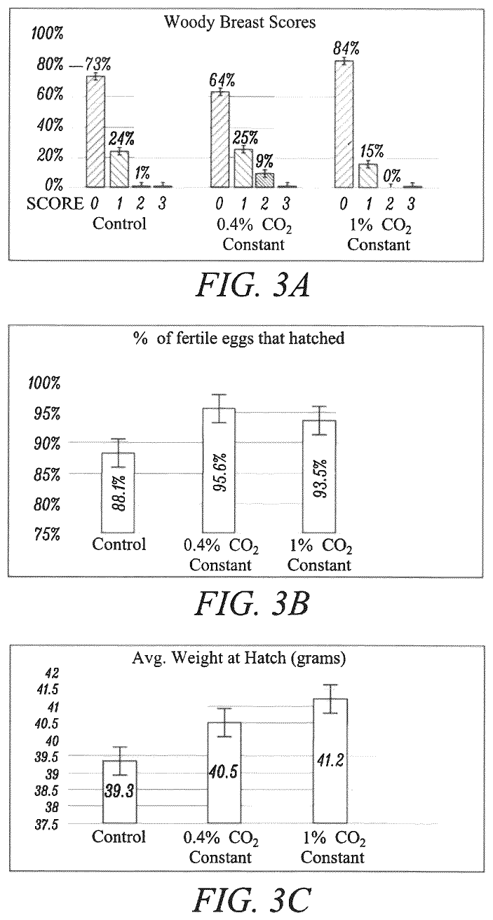

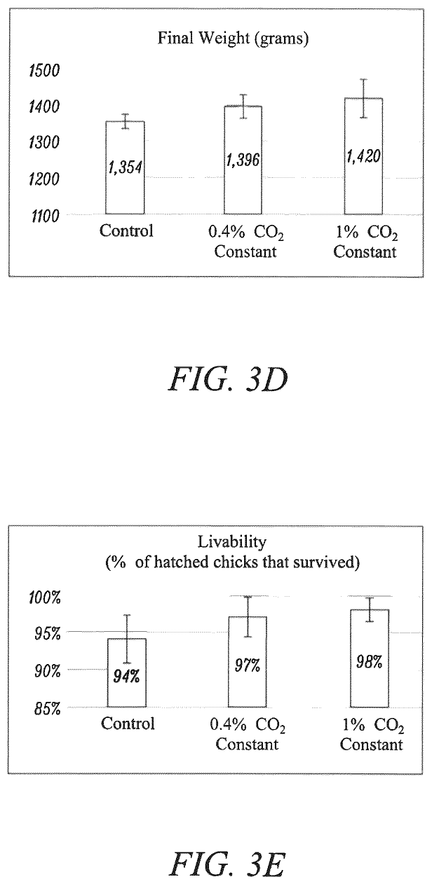

Results: FIGS. 3A, 3B, 3C, 3D, and 3E compare various parameters for chicken that were hatched from eggs that were incubated in CO₂ conditions where no external CO₂ was injected into the incubator (referred to as ‘control’ birds) vs. chicken that were hatched from eggs that were subjected to constant CO₂ concentrations of 4,000 ppm in the incubator for 18 days (referred to as ‘4,000 ppm CO₂’ or ‘0.4% CO₂’ birds) vs. chicken that were hatched from eggs that were subjected to constant CO₂ concentrations of 10,000 ppm in the incubator for 18 days (referred to as ‘10,000 ppm CO₂’ or ‘1% CO₂’ birds).

FIG. 3A shows Woody Breast scores for chickens in the three batches. As can be seen, the CO₂ treatment according to Regimen 1 at 10,000 ppm (1% CO₂) significantly reduced the incidence of WB in the chickens compared to the control birds. The % of birds scoring 1 reduced from 24% to 15%. However, for the 0.4% CO₂ test batch, the WB scores were higher, indicating an increase in WB incidence. There was an increase in score 2 scores from 1% in control to 9% in the 0.4% CO₂ batch, which is significant. These results show that simply increasing the concentration of CO₂ in the incubator is not sufficient. Specific recipes are needed to improve the woody breast scores of the birds.

FIG. 3B compares the percent of fertile eggs that hatched at the end of 21 days of incubation. 88.1% of the control birds hatched at the end of the 21 days, whereas 95.6% of the 0.4% CO₂ test birds hatched, whereas 93.5% of the test 1% CO₂ eggs hatched. This is important since an increase in hatchability significantly improves the profitability of the operation.

FIG. 3C compares the average weight of the chicken just after they hatched. The average weight of the birds increased by approximately 5% due to the 1% CO₂ treatment in the incubator. FIG. 3D compares the final weight of the birds at 28 days. The average weight of the 1% CO₂ birds was approximately 5% higher than the control birds.

FIG. 3E compares the percentage of hatched birds that survived through to the end of the 28 days. Approximately 4% more of the 1% CO₂ birds survived compared to the control birds.

### Example—Regimen 2

### Experimental:

600 fertilized eggs from a commonly used commercial chicken strain, Ross 708, that were less than 1 week old since being laid, and laid in the same laying facility by layers (chickens) of the same age, were split equally into batches of 300 each. One batch, referred to as control batch, was incubated for 18 days in an atmosphere in which no additional CO₂ was injected into this atmosphere from any source outside the egg. For this batch, the CO₂ concentration inside the incubator varied between ambient atmospheric (i.e. about 409 ppm) and as high as 1,500 ppm (resulting from CO₂ generation by the egg). The other batch, referred to as test CO₂ ramp batch or 1% CO₂ ramp test batch, was incubated for 18 days according to Regimen 2 described herein, wherein the CO₂ concentration in the incubator for the first 24 hours was maintained at normal atmospheric conditions (i.e. approximately 409 ppm) by not injecting any additional external CO₂ and then every 24 hours the CO₂ concentration was increased for 24 hours by an equal amount such that on the 18^{th} day the CO₂ concentration was 10,000 ppm (i.e. 1% CO₂) by occasional injections of CO₂ as necessary. Both batches were incubated at approximately 100° F. and between 30% and 70% relative humidity. At the end of the eighteenth day of incubation, both batches of eggs were transferred to hatcher equipment, where the eggs hatched over the next 3 days. No CO₂ was injected into the hatcher from any source outside the eggs for this period of 3 days for either batch. Thus, the only difference between the conditions under which the two batches were incubated was that one batch was subjected to the Regimen 2 reaching a concentration of 10,000 ppm CO₂ concentration on the 18th day. During the incubation period the oxygen concentration in the control incubator was always above 20%, whereas in the 1% CO₂ ramp up incubators, the oxygen level was typically above 19%.

Once the chicks hatched, approximately 150 of the male chicks from each batch were taken to grow cages and they began to be fed water and feed. From this point onwards, there was no difference in the treatment of the birds. 28 days after hatching the chickens from each batch were weighed and then stunned. Then the birds were measured for Woody Breast scores by manual palpitation by a trained panel of experts under single-blinded conditions. Each breast was given a score of either 0, 1, 2 or 3. Score 0 means no WB was detected. Score 3 meant very high levels of WB detected and scores 1 and 2 meant intermediate levels of WB were detected.

### Results:

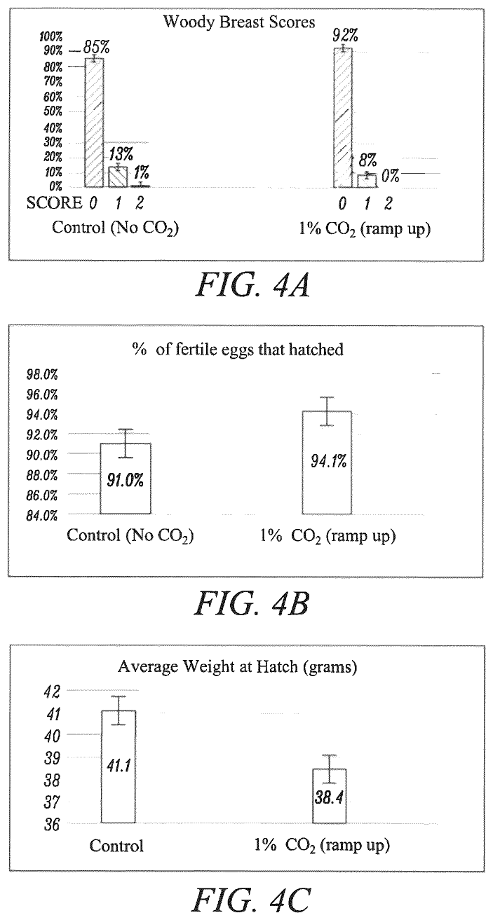

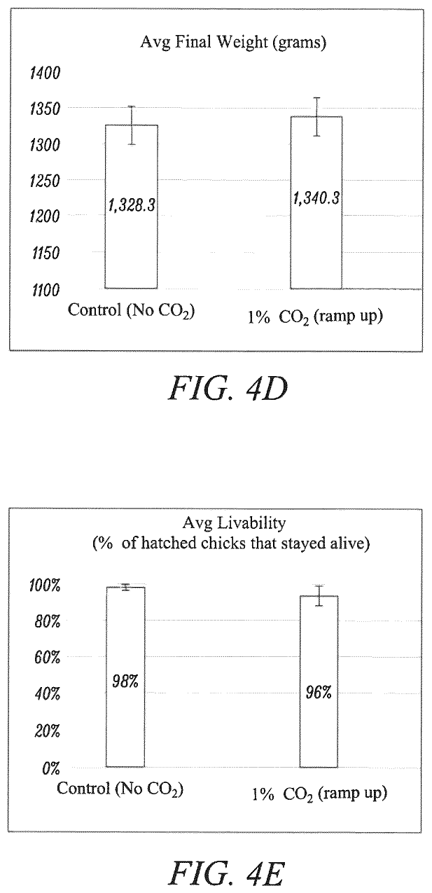

FIGS. 4A, 4B, 4C, 4D, and 4E compare various parameters for chicken that were hatched from eggs that were incubated in CO₂ conditions where no external CO₂ was injected into the incubator (referred to as ‘control’ birds) vs. chicken that were hatched from eggs that were subjected to ramp up CO₂ concentrations reaching 10,000 ppm in the incubator on day 18 (referred to as ‘10,000 ppm CO₂ ramp up’ or ‘1% CO₂ ramp up’ birds).

FIG. 4A shows Woody Breast scores for chickens which as eggs being incubated had been subjected to ramp up CO₂ concentrations reaching 10,000 ppm CO₂ in the incubator, or atmospheric CO₂ concentration with no injection of additional CO₂ into the incubator from any source outside of the egg. As can be seen, the CO₂ treatment according to Regimen 2 significantly reduced the incidence of WB in the chickens. The % of birds scoring 1 reduced from 13% to 8%.

FIG. 4B compares the percent of fertile eggs that hatched at the end of 21 days of incubation. 91% of the control birds hatched at the end of the 21 days, whereas 94.1% of the test 1% CO₂ ramp up eggs hatched. This is important since an increase in hatchability significantly improves the profitability of the operation.

FIG. 4C compares the average weight of the chicken just after they hatched. The average weight of the birds at hatch was approximately 6.5% lower for the 1% CO₂ ramp up treatment in the incubator compared to the control batch. However as seen in FIG. 4D, the 1% CO₂ ramp up birds quickly put on weight during the next 28 days, ending up slightly heavier than the control birds.

FIG. 4E shows the there was no statistical difference in the percentage of birds that survived, although the average livability was slightly higher in the control birds.

### Example—Regimen 3

### Experimental:

600 fertilized eggs from a commonly used commercial chicken strain, Ross 708, that were less than 1 week old since being laid, and laid in the same laying facility by layers (chickens) of the same age, were split equally into batches of 300 each. One batch, referred to as control batch, was incubated for 18 days in an atmosphere in which no additional CO₂ was injected into this atmosphere from any source outside the egg. For this batch, the CO₂ concentration inside the incubator varied between normal atmospheric (i.e. about 409 ppm) and as high as 1,500 ppm (resulting from CO₂ generation by the egg). The other batch, referred to as 7,500 ppm test CO₂ on-off batch or 0.75% CO₂ on-off test batch, was incubated for 18 days according to Regime 3 described herein, wherein the CO₂ concentration in the incubator for the first 24 hours was maintained constant at 7,500 ppm, followed by the next 24 hours where no external CO₂ was injected into the incubator (and the only CO₂ in the incubator came from the normal atmosphere and the CO₂ generation from the eggs). These two 24 hour periods were then continuously repeated till the end of day 18. During the CO₂ off periods, the CO₂ concentration in the incubator was between 409 ppm and 1,500 ppm. Both batches were incubated at approximately 100° F. and between 30% and 70% relative humidity. At the end of the eighteenth day of incubation, both batches of eggs were transferred to hatcher equipment, where the eggs hatched over the next 3 days. No CO₂ was injected into the hatcher from any source outside the eggs for this period of 3 days. Thus, the only difference between the conditions under which the two batches were incubated was that one batch was subjected to the Regimen 3 reaching a concentration of 10,000 ppm CO₂ concentration on the 18th day. During the incubation period the oxygen concentration in the control incubator was always above 20%, whereas in the 0.75% CO₂ on-off incubators, the oxygen level was typically above 19%.

Once the chicks hatched, approximately 150 of the male chicks from each batch were taken to grow cages and they began to be fed water and feed. From this point onwards, there was no difference in the treatment of the birds. 28 days after hatching the chickens from each batch were weighed and then stunned. Then the birds were measured for Woody Breast scores by manual palpitation by a trained panel of experts under single-blinded conditions. Each breast was given a score of either 0, 1, 2 or 3. Score 0 means no WB was detected. Score 3 meant very high levels of WB detected and scores 1 and 2 meant intermediate levels of WB were detected.

### Results:

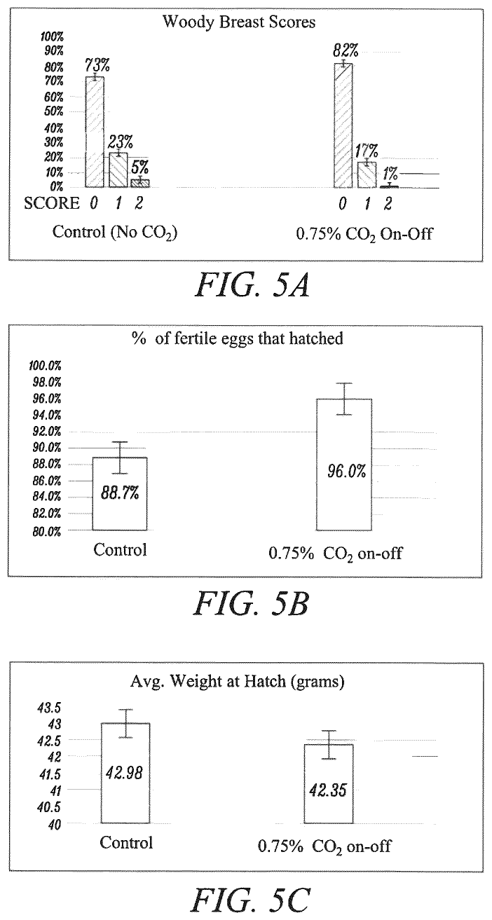

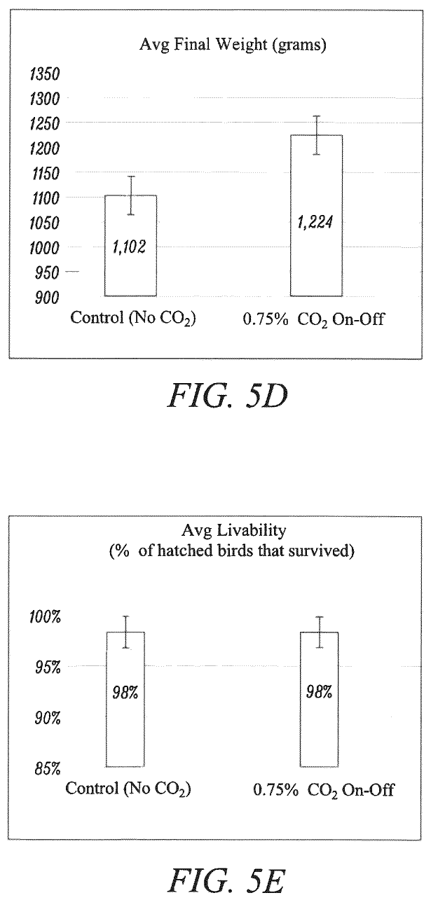

FIGS. 5A, 5B, 5C, 5D, and 5E compare various parameters for chicken that were hatched from eggs that were incubated in CO₂ conditions where no external CO₂ was injected into the incubator (referred to as ‘control’ birds) vs. chicken that were hatched from eggs that were subjected to on-off CO₂ concentrations reaching 7,500 ppm on alternate days till day 18 (referred to as ‘7,500 ppm CO₂ on-off’ or ‘0.75% CO₂ on-off’ birds).

FIG. 5A compares Woody Breast scores of the control and 0.75% CO₂ on-off birds. As can be seen, the CO₂ treatment according to Regimen 3 significantly reduced the incidence of WB in the chickens. The % of birds scoring 1 reduced from 23% to 17%, scoring 2 reduced from 5% to 1%, while birds scoring 0 increased from 73% to 82% on average.

FIG. 5B compares the percent of fertile eggs that hatched at the end of 21 days of incubation. 88.7% of the control birds hatched at the end of the 21 days, whereas 96% of the test 0.75% CO₂ on-off eggs hatched. This is important since an increase in hatchability significantly improves the profitability of the operation.

FIG. 5C compares the average weight of the chicken just after they hatched. The average weight of the birds at hatch was approximately 1.5% lower due to the 0.75% CO₂ on-off treatment in the incubator as compared to the control batch. However as seen in FIG. 5D, the 0.75% CO₂ on-off birds quickly put on weight during the next 28 days, ending up 11% heavier than the control birds.

Finally, FIG. 4E shows the there was no statistical difference in the percentage of birds that survived.

### Example 2

This example illustrates that oxygen-containing nanobubbles in water administered to hatched chicks reduces onset of woody breast disorder.

Oxygen nanobubbles were generated in tap water using pure oxygen gas and a pressure-dissolution based nanobubble generator. No NB stability enhancing chemicals or components we added to the water. The concentration of oxygen nanobubbles generated was approximately 100 million bubbles per ml of tap water. This concentration was measured via NTA after subtracting for the initial nanobubble concentration in the tap water.

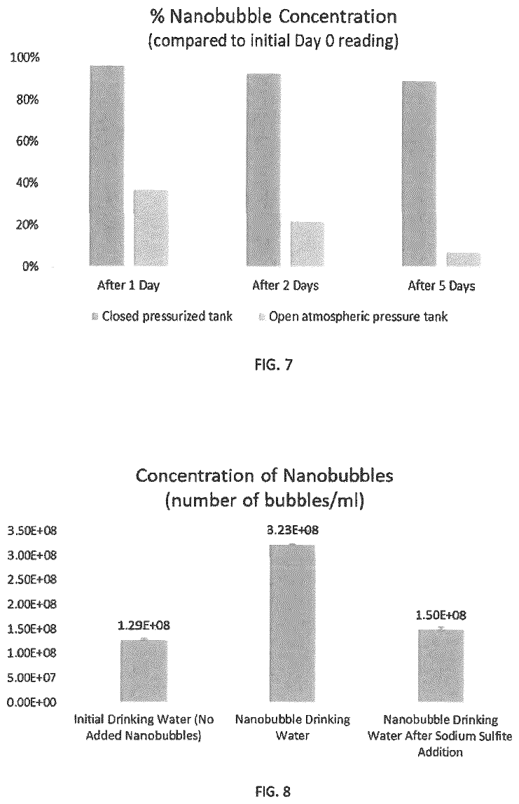

The oxygen nanobubble water was split into two halves. One half was kept in a closed tank that had a very slight headspace pressure of approximately 1 psig. The other half was kept in an open vessel at atmospheric pressure. Samples were taken from these two different tanks at day 1, 2 and 5 after start of storage. These samples were analyzed for nanobubble concentration using NTA. The graph in FIG. 7 shows the concentration of nanobubbles expressed as % compared to the initial nanobubble concentration. The oxygen-containing nanobubbles stored in the closed vessel under slight pressure lasted much longer than the nanobubbles stored in an open tank at atmospheric pressure. This shows the importance of (1) keeping the water in which oxygen-containing nanobubbles had been generated under at least slight pressure in a closed tank and pipeline until the chickens drink the water and (2) regenerating the oxygen-containing nanobubbles in the water at least every 12 hours to ensure that the chickens are getting the required concentration of oxygen-containing nanobubbles.

The following process was used in the experiments reported in this Example. This is the preferred, but not the only, method of generating nanobubbles.

A closed tank (or similar device) maintained at higher than atmospheric pressure is inserted in the regular drinking water line. A recirculation loop that includes a recirculating pump and a nanobubble generator is inserted around the tank. When the recirculating pump is run, the water from the tank flows through the nanobubble generator. At the same time oxygen gas is flowed into the nanobubble generator. The nanobubble generator takes the oxygen gas and creates oxygen nanobubbles in the recirculating water. The water with the oxygen nanobubbles is returned back to the water tank.

The recirculation loop is run for a specified period of time to make sure that the desired characteristics of oxygen nanobubbles are produced. Typically the water in the tank has to be recirculated through the nanobubble generator at least 2 times, more preferably at least 5 times and most preferably at least 20 times. This also creates a relation between flow rate and time for which the recirculation loop is run to create the nanobubbles. For example, if the capacity of the tank is 100 gallons, and the flow rate of the recirculation loop pump is 5 gallons per minute, then the recirculation loop should be run at least 40 minutes.

The tank is typically sized at 1.5 times the maximum amount of water that all the full size birds can drink in one day. The flow rate of the recirculation loop can be varied, and usually depends on the type and design of the nanobubble generator.

The temperature of the water should be maintained within 5 degrees Fahrenheit of the incoming drinking water. Preferably the water containing oxygen-containing nanobubbles should be within 5 F of room temperature. As noted above, preferred temperatures are in the range of 35-120 F, more preferably 50-100 F. This is important because in some cases, the recirculation through the nanobubble generator can cause the temperature of the water to increase. As the temperature of the water increases, its capacity to hold oxygen-containing nanobubbles and dissolved oxygen goes down, negatively affecting its efficacy.

Ross 708 (a species of chicken that is usually grown to heavy weights) male chicks from a commercial hatchery were used for the tests. 200 male newly hatched chicks were randomly divided into two equal groups. 100 male chicks were grown while providing them control unoxygenated city drinking water from day 1 (after they hatched) to day 28 of their growth and another 100 male chicks were grown while providing them drinking water containing oxygen-enriched nanobubbles from day 1 to day 28 of their growth. Standard nipple drinkers were used to make drinking water available for both sets of chickens. Both sets of chickens were grown for 28 days. In both cases, the drinking water was always kept pressurized in the water line supplying water to the nipples 20. The chickens were free to drink as much water as they wanted by tapping a nipple 20 to make the water flow. Everything else was maintained the same for both sets of chickens, including feed (other than the water), lighting protocol, and temperatures.

Nanobubbles of oxygen were created in the test drinking water by means of a pressure dissolution—expansion method described above. The city drinking water was pressurized and flowed through a chamber in which oxygen gas was added to the water. The oxygen content of the gas used was 99.99% purity oxygen. The chamber was designed to ensure that all the oxygen that was fed was dissolved into the water. At the outlet of the chamber, the pressure of the water with dissolved oxygen was dropped in a controlled manner so as to allow some of the dissolved oxygen to come out of solution in the form of nanobubbles. This drinking water containing oxygen-enriched nanobubbles was fed to the 100 test birds.

Analysis by NTA of the original drinking water showed that the drinking water contained approximately 129 million bubbles (or particles) per ml of water. After generation of oxygen nanobubbles in the drinking water, a sample of the drinking water with the oxygen-enriched nanobubbles measured by NTA showed 323 million nanobubbles/ml of water. The difference between the two readings showed that approximately 194 million oxygen-enriched nanobubbles were generated per ml of drinking water. Finally, a sample of drinking water with oxygen-enriched nanobubbles was subjected to excessive sodium sulfite solution addition in presence of cobalt catalyst. This addition of sodium sulfite destroyed all the oxygen-enriched nanobubbles after a period of waiting. This sample was then analyzed by NTA which showed approximately 150 million bubbles/particles per ml of drinking water, which is similar to the original 129 million/ml. Thus at least 173 million oxygen-enriched nanobubbles were generated per ml of drinking water and fed to the chicken. These figures are depicted in the bar graphs in FIG. 8.

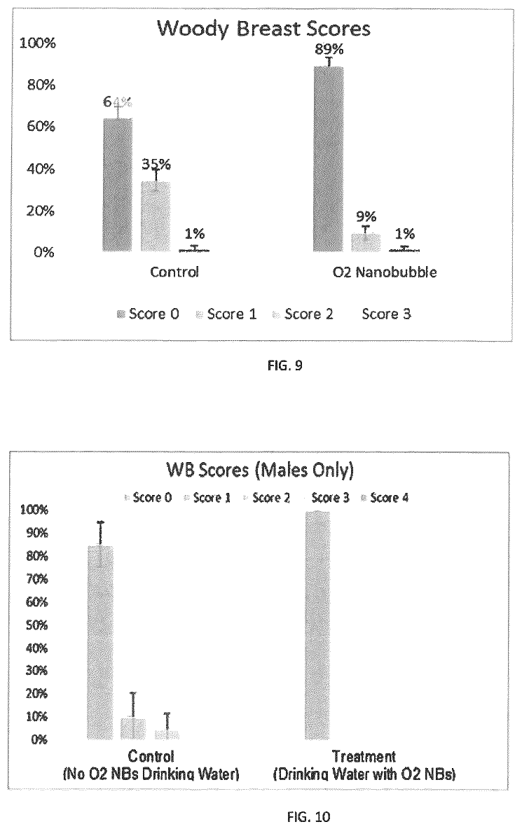

After 28 days the chickens were evaluated for WB using a trained sensory panel that evaluated the breasts of all 200 chicken from both treatments. In the graph in FIG. 9, a score of 0 indicates no WB was found, a score of 1 indicates mild WB, a score of 2 indicates a medium level of WB, and a score of 3 indicates a very high level of WB. The number of chickens showing a score of 1 or higher than 1 dropped by 72% for the chickens that had been fed water containing oxygen-enriched nanobubbles. There were no differences measured in daily weight gains, final weights, food conversion ratios, mortalities etc. between the two sets of birds.

In a second trial the same parameters as above were used, with the exception that the chickens were grown for 42 days instead of 28 days. In this trial the incidence of WB was completely eliminated, as is shown in FIG. 10. There were no differences measured in daily weight gains, final weights, food conversion ratios, mortalities etc. between the two sets of birds.

What is claimed is:

1. A method of reducing or eliminating the incidence of woody breast in poultry that has hatched from a fertilized unhatched poultry egg, comprising, before the poultry has hatched from the egg, incubating the egg for an incubation period of 18 to 21 consecutive days in a gaseous atmosphere which is in contact with the egg, and during that time, feeding carbon dioxide from a source outside the egg into the gaseous atmosphere as necessary so that for at least one period of time of at least 12 hours the carbon dioxide concentration in the gaseous atmosphere which is in contact with the egg is 7,500 ppm to 20,000 ppm.

2. A method according to claim 1, comprising incubating the egg for an incubation period of 18 to 21 consecutive days in a gaseous atmosphere which is in contact with the egg, and during that time, feeding carbon dioxide from a source outside the egg into the gaseous atmosphere as necessary so that for at least one period of time of at least 24 hours the carbon dioxide concentration in the gaseous atmosphere which is in contact with the egg is 7,500 ppm to 20,000 ppm.

3. A method according to claim 1, comprising incubating the egg for an incubation period of 18 to 21 consecutive days in a gaseous atmosphere which is in contact with the egg, and during that time, feeding carbon dioxide from a source outside the egg into the gaseous atmosphere as necessary so that for at least six periods of time of at least 12 hours the carbon dioxide concentration in the gaseous atmosphere which is in contact with the egg is 7,500 ppm to 20,000 ppm.

4. A method according to claim 1, comprising exposing the fertilized unhatched poultry egg for at least 1 day of the first 18 days of the incubation period to a gaseous atmosphere which is in contact with the egg and which contains carbon dioxide at an incubation concentration that is within 10% of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of a set value between 7,500 ppm CO₂ and 20,000 ppm CO₂.

5. A method according to claim 4, comprising exposing the fertilized unhatched poultry egg for at least 6 days of the first 18 days of the incubation period to a gaseous atmosphere which is in contact with the egg and which contains carbon dioxide at an incubation concentration that is within 10%, and preferably within 5%, of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of a set value between 7,500 ppm CO₂ and 20,000 ppm CO₂.

6. A method according to claim 4 wherein the fertilized unhatched poultry egg is a chicken egg.

7. A method according to claim 6, comprising
exposing the fertilized unhatched chicken egg for at least 12 days of the first 18 days of the incubation period to a gaseous atmosphere which is in contact with the egg and which contains carbon dioxide at an incubation concentration that is within 10% of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of a set value between 7,500 ppm CO₂ and 20,000 ppm CO₂.

8. A method according to claim 6, comprising
exposing the fertilized unhatched chicken egg throughout the first 18 days of the incubation period to a gaseous atmosphere which is in contact with the egg and which contains carbon dioxide at an incubation concentration that is within 10% of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of a set value between 7,500 ppm CO₂ and 20,000 ppm CO₂.

9. A method according to claim 8 wherein the set value throughout the first 18 days is constant for all 18 days.

10. A method according to claim 8 wherein the egg is incubated for a period of 18 consecutive days in a gaseous atmosphere which is in contact with the egg and which contains carbon dioxide at an incubation concentration that is within 5% of a set value between 7,500 ppm CO₂ and 15,000 ppm CO₂.

11. A method according to claim 8 wherein the egg is incubated for a period of 18 consecutive days in a gaseous atmosphere which is in contact with the egg and which contains carbon dioxide at an incubation concentration that is within 10% of a set value of 10,000 ppm CO₂.

12. A method according to claim 8 wherein the egg is incubated for a period of 18 consecutive days in a gaseous atmosphere which is in contact with the egg and which contains carbon dioxide at an incubation concentration that is within 5% of a set value of 10,000 ppm CO₂.

13. A method according to claim 8 wherein for 3 consecutive days immediately following the 18-day period the egg is incubated in an atmosphere into which CO₂ is fed from a source outside the egg as necessary to maintain the CO₂ concentration of the atmosphere in contact with the egg at a concentration that is within 5% of a set value between 7,500 ppm CO₂ and 15,000 ppm CO₂.

14. A method according to claim 8 wherein for 3 consecutive days immediately following the 18-day period the egg is incubated in an atmosphere into which CO₂ is fed from a source outside the egg as necessary to maintain the CO₂ concentration of the atmosphere in contact with the egg at a concentration that is within 10% of a set value of 10,000 ppm CO₂.

15. A method according to claim 8 wherein for 3 consecutive days immediately following the 18-day period the egg is incubated in an atmosphere into which CO₂ is fed from a source outside the egg as necessary to maintain the CO₂ concentration of the atmosphere in contact with the egg at a concentration that is within 5% of a set value of 10,000 ppm CO₂.

16. A method according to claim 1, comprising
exposing the fertilized unhatched poultry egg to a gaseous atmosphere which is in contact with the egg for consecutive incremental periods in each of which incremental periods the carbon dioxide concentration in the gaseous atmosphere is maintained at a value that is within 10% of a set value that is constant throughout the incremental period and is between the CO₂ concentration of the ambient atmosphere and 20,000 ppm CO₂ by feeding carbon dioxide into the gaseous atmosphere from a source outside the egg as necessary to maintain the CO₂ concentration in the gaseous atmosphere within 10% of said set value, wherein the set values and the CO₂ concentrations in the gaseous atmosphere increase from each of said incremental periods to the next incremental period, such that in the incremental period at the end of the incubation period the CO₂ concentration in the gaseous atmosphere is within 10% of a value between 7,500 ppm CO₂ and 20,000 ppm CO₂, and then for 3 consecutive days immediately following the first 18 days of the incubation period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of a constant set value between 7,500 ppm CO₂ and 20,000 ppm CO₂.

17. A method according to claim 16 wherein each of said incremental periods is 18 to 24 hours in duration and there are 2 to 20 incremental periods.

18. A method according to claim 16 wherein each set value increases from one incremental period to the next incremental period by equal increments.

19. A method according to claim 16 wherein the fertilized unhatched poultry egg is a chicken egg.

20. A method according to claim 19 wherein each of said incremental periods is 18 to 24 hours in duration and there are 2 to 20 incremental periods.

21. A method according to claim 19 wherein each set value increases from one incremental period to the next incremental period by equal increments.

22. A method according to claim 19 wherein the egg is incubated for 18 consecutive incremental periods of 24 hours each in a gaseous atmosphere which is in contact with the egg, in each of which periods the carbon dioxide concentration in the gaseous atmosphere is maintained at a value that is within 5% of a set value that is constant throughout the period and is between the CO₂ concentration of the ambient atmosphere and 15,000 ppm CO₂ by feeding carbon dioxide into the gaseous atmosphere from a source outside the egg as necessary to maintain the CO₂ concentration in the gaseous atmosphere within 5% of said set value.

23. A method according to claim 22 wherein the set values and the CO₂ concentrations in the gaseous atmosphere increase from each 24-hour period to the next 24-hour period such that in the eighteenth 24-hour period the CO₂ concentration in the gaseous atmosphere is within 5% of a value between 7,500 ppm CO₂ and 15,000 ppm CO₂.

24. A method according to claim 22 wherein the set values and the CO₂ concentrations in the gaseous atmosphere increase from each 24-hour period to the next 24-hour period such that in the eighteenth 24-hour period the CO₂ concentration in the gaseous atmosphere is within 10% of 10,000 ppm CO₂.

25. A method according to claim 22 wherein the set values and the CO₂ concentrations in the gaseous atmosphere increase from each 24-hour period to the next 24-hour period such that in the eighteenth 24-hour period the CO₂ concentration in the gaseous atmosphere is within 5% of 10,000 ppm CO₂.

26. A method according to claim 22 wherein for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 5% of a constant set value between 7,500 ppm CO₂ and 15,000 ppm CO₂.

27. A method according to claim 22 wherein for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10% of 10,000 ppm CO₂.

28. A method according to claim 22 wherein for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 5% of 10,000 ppm CO₂.

29. A method according to claim 1, comprising, throughout the incubation period, alternatingly
(A) exposing the fertilized unhatched poultry egg to a gaseous atmosphere which is in contact with the egg for an exposure period, in each of which exposure periods the carbon dioxide concentration in the gaseous atmosphere is maintained at a value that is within 10% of a set value between 7,500 ppm CO₂ and 20,000 ppm CO₂ that is constant in each exposure period, by feeding carbon dioxide into the gaseous atmosphere from a source outside the egg as necessary to maintain the CO₂ concentration in the gaseous atmosphere in the exposure period within 10% of said set value, wherein the set value is the same in all of the exposure periods, and
(B) exposing the fertilized unhatched egg for an intervening period to a gaseous atmosphere into which no carbon dioxide is fed into the gaseous atmosphere from outside the egg.

30. A method according to claim 29, wherein each exposure period is 18 to 24 hours in duration and each intervening period is 18 to 24 hours in duration.

31. A method according to claim 29, wherein there are 2 to 20 exposure periods and 2 to 20 intervening periods.

32. A method according to claim 29 wherein the fertilized unhatched poultry egg is a chicken egg.

33. A method according to claim 32, wherein each exposure period is 18 to 24 hours in duration and each intervening period is 18 to 24 hours in duration.

34. A method according to claim 32, wherein there are 2 to 20 exposure periods and 2 to 20 intervening periods.

35. A method according to claim 32 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 5% of a set value between 7,500 ppm CO₂ and 15,000 ppm CO₂ that is constant in each exposure period, by feeding carbon dioxide into the gaseous atmosphere from a source outside the egg as necessary to maintain the CO₂ concentration in the gaseous atmosphere within 5% of said set value.

36. A method according to claim 32 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 10% of a set value of 10,000 ppm CO₂ that is constant in each exposure period, by feeding carbon dioxide into the gaseous atmosphere from a source outside the egg as necessary to maintain the CO₂ concentration in the gaseous atmosphere within 10% of said set value.

37. A method according to claim 32 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 5% of a set value of 10,000 ppm CO₂ that is constant in each exposure period, by feeding carbon dioxide into the gaseous atmosphere from a source outside the egg as necessary to maintain the CO₂ concentration in the gaseous atmosphere within 5% of said set value.

38. A method according to claim 1, comprising, throughout the incubation period, alternatingly
(A) exposing the fertilized unhatched poultry egg to a gaseous atmosphere which is in contact with the egg for an exposure period, in each of which exposure periods the carbon dioxide concentration in the gaseous atmosphere is maintained at a value that is within 10% of a set value between the CO₂ concentration of the ambient atmosphere and 20,000 ppm CO₂ that increases from one exposure period to the next exposure period, by feeding carbon dioxide into the gaseous atmosphere from a source outside the egg as necessary to maintain the CO₂ concentration in the gaseous atmosphere in the exposure period within 10% of said set value, such that in the last incremental period the CO₂ concentration in the gaseous atmosphere is within 10% of a value between 7,500 ppm CO₂ and 20,000 ppm CO₂ and
(B) exposing the fertilized unhatched egg for an intervening period of to a gaseous atmosphere into which no carbon dioxide is fed into the gaseous atmosphere from outside the egg.

39. A method according to claim 38, wherein each exposure period is 18 to 24 hours in duration and each intervening period is 18 to 24 hours in duration.

40. A method according to claim 38 wherein each first set value increases from one exposure period to the next exposure period by equal increments.

41. A method according to claim 38 wherein the fertilized unhatched poultry egg is a chicken egg.

42. A method according to claim 41, wherein each exposure period is 18 to 24 hours in duration and each intervening period is 18 to 24 hours in duration.

43. A method according to claim 41 wherein each first set value increases from one exposure period to the next exposure period by equal increments.

44. A method according to claim 41 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 5% of a first set value between the CO₂ concentration of the ambient atmosphere and 20,000 ppm CO₂ by feeding carbon dioxide into the gaseous atmosphere from a source outside the egg as necessary to maintain the CO₂ concentration in the gaseous atmosphere within 5% of said first set value.

45. A method according to claim 41 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 10% of a first set value between the CO₂ concentration of the ambient atmosphere and 20,000 ppm CO₂ by feeding carbon dioxide into the gaseous atmosphere from a source outside the egg as necessary to maintain the CO₂ concentration in the gaseous atmosphere within 10% of said first set value.

46. A method according to claim 41 wherein the egg is incubated for 9 to 11 exposure periods and 9 to 11 intervening periods in a gaseous atmosphere which is in contact with the egg, wherein in each exposure period the carbon dioxide concentration in the gaseous atmosphere is maintained at a value that is within 5% of a first set value between the CO₂ concentration of the ambient atmosphere and 15,000 ppm CO₂ by feeding carbon dioxide into the gaseous atmosphere from a source outside the egg as necessary to maintain the CO₂ concentration in the gaseous atmosphere within 5% of said first set value.

47. A method of reducing or eliminating the incidence of woody breast in poultry that has hatched from a fertilized unhatched poultry egg, comprising, before the poultry has hatched from the egg, incubating the egg for an incubation period of 18 to 21 consecutive days in a gaseous atmosphere which is in contact with the egg, and during that time, feeding carbon dioxide from a source outside the egg into the gaseous atmosphere as necessary so that for at least one period of time of at least 12 hours the carbon dioxide concentration in the gaseous atmosphere which is in contact with the egg is 7,500 ppm to 20,000 ppm,
and then
administering enterally to the poultry water comprising in the water nanobubbles which comprise oxygen.

48. A method according to claim 47 wherein the fertilized unhatched poultry egg is a chicken egg.

49. A method according to claim 48 wherein the water containing said nanobubbles comprises 50 vol. % to 100 vol. % oxygen.

50. A method according to claim 48 wherein said water containing nanobubbles that is administered enterally to the chicken is maintained at a pressure of up to 25 psig until the chicken ingests it.

51. A method according to claim 48 wherein at least 5% of all of the water that is administered enterally to the chicken comprises said nanobubbles.

52. A method according to claim 48 wherein 50 vol. % to 100 vol. % of all of the water that is administered enterally to the chicken comprises said nanobubbles.

53. A method according to claim 48 wherein the average diameter of said nanobubbles is 10 to 1000 nanometers.

54. A method according to claim 48 wherein the water administered to the chicken comprises at least 100 million of said nanobubbles per milliliter of water.

55. A method according to claim 48 wherein the aggregate amount of oxygen in said water administered to the chicken is 20 to 150 milligrams of oxygen per liter of said water.

56. A method according to claim 48 wherein the enteral administration of said water to the chicken begins no longer than 2 weeks after the chicken has hatched.

57. A method according to claim 48 wherein the enteral administration of said water to the chicken begins no longer than 2 days after the chicken has hatched.

58. A method according to claim 47, comprising
exposing the fertilized unhatched poultry egg for at least 1 day of the first 18 days of the incubation period to a gaseous atmosphere which is in contact with the egg and which contains carbon dioxide at an incubation concentration that is within 10%, and preferably within 5%, of a set value between 7,500 ppm CO₂ and 20,000 ppm CO₂, and during the 18-day period feeding carbon dioxide into the atmosphere as necessary to maintain the CO₂ concentration in which the egg is incubated at said incubation concentration, and then for 3 consecutive days immediately following the 18-day period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10%, and preferably within 5%, of a set value between 7,500 ppm CO₂ and 20,000 ppm CO₂.

59. A method according to claim 58 wherein the fertilized unhatched poultry egg is a chicken egg.

60. A method according to claim 59 wherein the water containing said nanobubbles comprises 50 vol. % to 100 vol. % oxygen.

61. A method according to claim 59 wherein said water containing nanobubbles that is administered enterally to the chicken is maintained at a pressure of up to 25 psig until the chicken ingests it.

62. A method according to claim 59 wherein at least 5% of all of the water that is administered enterally to the chicken comprises said nanobubbles.

63. A method according to claim 59 wherein 50 vol. % to 100 vol. % of all of the water that is administered enterally to the chicken comprises said nanobubbles.

64. A method according to claim 59 wherein the average diameter of said nanobubbles is 10 to 1000 nanometers.

65. A method according to claim 59 wherein the water administered to the chicken comprises at least 100 million of said nanobubbles per milliliter of water.

66. A method according to claim 59 wherein the aggregate amount of oxygen in said water administered to the chicken is 20 to 150 milligrams of oxygen per liter of said water.

67. A method according to claim 59 wherein the enteral administration of said water to the chicken begins no longer than 2 weeks after the chicken has hatched.

68. A method according to claim 59 wherein the enteral administration of said water to the chicken begins no longer than 2 days after the chicken has hatched.

69. A method according to claim 47, comprising
exposing the fertilized unhatched poultry egg to a gaseous atmosphere which is in contact with the egg for consecutive incremental periods in each of which incremental periods the carbon dioxide concentration in the gaseous atmosphere is maintained at a value that is within 10%, and preferably within 5%, of a set value that is constant throughout the incremental period and is between the CO₂ concentration of the ambient atmosphere and 20,000 ppm CO₂ by feeding carbon dioxide into the gaseous atmosphere from a source outside the egg as necessary to maintain the CO₂ concentration in the gaseous atmosphere within 10%, and preferably within 5%, of said set value, wherein the set values and the CO₂ concentrations in the gaseous atmosphere increase from each of said incremental periods to the next incremental period, such that in the incremental period at the end of the incubation period the CO₂ concentration in the gaseous atmosphere is within 10%, and preferably within 5%, of a value between 7,500 ppm CO₂ and 20,000 ppm CO₂, and then for 3 consecutive days immediately following the first 18 days of the incubation period incubating the egg in an atmosphere into which no additional CO₂ is fed from any source outside the egg or into which CO₂ is fed from a source outside the egg as necessary to maintain the CO₂ concentration of the atmosphere in contact with the egg at a concentration that is within 10%, and preferably within 5%, of a constant set value between 7,500 ppm CO₂ and 20,000 ppm CO₂.

70. A method according to claim 69 wherein the fertilized unhatched poultry egg is a chicken egg.

71. A method according to claim 70 wherein the water containing said nanobubbles comprises 50 vol. % to 100 vol. % oxygen.

72. A method according to claim 70 wherein said water containing nanobubbles that is administered enterally to the chicken is maintained at a pressure of up to 25 psig until the chicken ingests it.

73. A method according to claim 70 wherein at least 5% of all of the water that is administered enterally to the chicken comprises said nanobubbles.

74. A method according to claim 70 wherein 50 vol. % to 100 vol. % of all of the water that is administered enterally to the chicken comprises said nanobubbles.

75. A method according to claim 70 wherein the average diameter of said nanobubbles is 10 to 1000 nanometers.

76. A method according to claim 70 wherein the water administered to the chicken comprises at least 100 million of said nanobubbles per milliliter of water.

77. A method according to claim 70 wherein the aggregate amount of oxygen in said water administered to the chicken is 20 to 150 milligrams of oxygen per liter of said water.

78. A method according to claim 70 wherein the enteral administration of said water to the chicken begins no longer than 2 weeks after the chicken has hatched.

79. A method according to claim 70 wherein the enteral administration of said water to the chicken begins no longer than 2 days after the chicken has hatched.

80. A method according to claim 47, comprising, throughout the incubation period, alternatingly
(A) exposing the fertilized unhatched poultry egg to a gaseous atmosphere which is in contact with the egg for an exposure period, in each of which exposure periods the carbon dioxide concentration in the gaseous atmosphere is maintained at a value that is within 10%, and preferably within 5%, of a set value between 7,500 ppm CO₂ and 20,000 ppm CO₂ that is constant in each exposure period, by feeding carbon dioxide into the gaseous atmosphere from a source outside the egg as necessary to maintain the CO₂ concentration in the gaseous atmosphere in the exposure period within 10%, and preferably within 5%, of said set value, wherein the set value is the same in all of the exposure periods, and
(B) exposing the fertilized unhatched egg for an intervening period to a gaseous atmosphere into which no carbon dioxide is fed into the gaseous atmosphere from outside the egg.

81. A method according to claim 80 wherein the fertilized unhatched poultry egg is a chicken egg.

82. A method according to claim 81 wherein the water containing said nanobubbles comprises 50 vol. % to 100 vol. % oxygen.

83. A method according to claim 81 wherein said water containing nanobubbles that is administered enterally to the chicken is maintained at a pressure of up to 25 psig until the chicken ingests it.

84. A method according to claim 81 wherein at least 5% of all of the water that is administered enterally to the chicken comprises said nanobubbles.

85. A method according to claim 81 wherein 50 vol. % to 100 vol. % of all of the water that is administered enterally to the chicken comprises said nanobubbles.

86. A method according to claim 81 wherein the average diameter of said nanobubbles is 10 to 1000 nanometers.

87. A method according to claim 81 wherein the water administered to the chicken comprises at least 100 million of said nanobubbles per milliliter of water.

88. A method according to claim 81 wherein the aggregate amount of oxygen in said water administered to the chicken is 20 to 150 milligrams of oxygen per liter of said water.

89. A method according to claim 81 wherein the enteral administration of said water to the chicken begins no longer than 2 weeks after the chicken has hatched.

90. A method according to claim 81 wherein the enteral administration of said water to the chicken begins no longer than 2 days after the chicken has hatched.

91. A method according to claim 47, comprising, throughout the incubation period, alternatingly
(A) exposing the fertilized unhatched poultry egg to a gaseous atmosphere which is in contact with the egg for an exposure period, in each of which exposure periods the carbon dioxide concentration in the gaseous atmosphere is maintained at a value that is within 10%, and preferably within 5%, of a set value between the CO₂ concentration of the ambient atmosphere and 20,000 ppm CO₂ that increases from one exposure period to the next exposure period, by feeding carbon dioxide into the gaseous atmosphere from a source outside the egg as necessary to maintain the CO₂ concentration in the gaseous atmosphere in the exposure period within 10%, and preferably within 5%, of said set value, such that in the last incremental period the CO₂ concentration in the gaseous atmosphere is within 10%, and preferably within 5%, of a value between 7,500 ppm CO₂ and 20,000 ppm CO₂ and
(B) exposing the fertilized unhatched egg for an intervening period of to a gaseous atmosphere into which no carbon dioxide is fed into the gaseous atmosphere from outside the egg.

92. A method according to claim 91 wherein the fertilized unhatched poultry egg is a chicken egg.

93. A method according to claim 92 wherein the water containing said nanobubbles comprises 50 vol. % to 100 vol. % oxygen.

94. A method according to claim 92 wherein said water containing nanobubbles that is administered enterally to the chicken is maintained at a pressure of up to 25 psig until the chicken ingests it.

95. A method according to claim 92 wherein at least 5% of all of the water that is administered enterally to the chicken comprises said nanobubbles.

96. A method according to claim 92 wherein 50 vol. % to 100 vol. % of all of the water that is administered enterally to the chicken comprises said nanobubbles.

97. A method according to claim 92 wherein the average diameter of said nanobubbles is 10 to 1000 nanometers.

98. A method according to claim 92 wherein the water administered to the chicken comprises at least 100 million of said nanobubbles per milliliter of water.

99. A method according to claim 92 wherein the aggregate amount of oxygen in said water administered to the chicken is 20 to 150 milligrams of oxygen per liter of said water.

100. A method according to claim 92 wherein the enteral administration of said water to the chicken begins no longer than 2 weeks after the chicken has hatched.

101. A method according to claim 92 wherein the enteral administration of said water to the chicken begins no longer than 2 days after the chicken has hatched.
